(19) United States
(12) Patent Application Publication
(10) Pub. No.: US 2025/0099532 A1
(43) Pub. Date: Mar. 27, 2025

(54) METHODS OF TREATMENT USING OXYTOCIN

(71) Applicant: Belnap Pharmaceuticals, LLC, Encinitas, CA (US)

(72) Inventor: Drew Grant Belnap, Encinitas, CA (US)

(21) Appl. No.: 18/833,862

(22) PCT Filed: Jun. 30, 2022
(86) PCT No.: PCT/US2022/035860

§ 371 Date: Jul. 26, 2024

Related U.S. Application Data
(60) Provisional application No. 63/303,807, filed on Jan. 27, 2022.

(51) Int. Cl. A61K 38/095; A61K 31/135; A61K 31/407; A61K 31/57; A61K 31/58; A61P 25/22; A61P 25/24

(52) U.S. Cl. CPC A61K 38/095; A61K 31/135; A61K 31/407; A61K 31/57; A61K 31/58; A61P 25/22; A61P 25/24

(57) ABSTRACT

The present disclosure relates to methods for treating and/or preventing diseases and disorders using oxytocin targeting agents, and compositions relating to the same.

### CROSS REFERENCE TO RELATED 5 APPLICATIONS

This application claims the benefit under 35 U.S.C. § 119(e) of U.S. Provisional Application No. 63/303,807, filed Jan. 27, 2022, which application is incorporated by reference in its entirety.

### FIELD

The present disclosure relates to methods for treating and/or preventing diseases and disorders using oxytocin targeting agents, and compositions relating to the same.

### BACKGROUND

It has been estimated that 1 in 20 U.S. adults experience serious mental illness each year. Treatment choices for mental health conditions will vary from person to person. Even people with the same diagnosis will have different experiences, needs, goals and objectives for treatment. There is no “one size fits all” treatment. For severe patients, the options for treatment and success rates can be limited, and recovery rates low. People with serious mental illness are nearly twice as likely to develop cardiovascular and metabolic diseases than the general population, experience a higher rate of unemployment, are more likely to drop out of school, are more likely to develop a substance use disorder, are more likely to become hospitalized, and even are more likely to become incarcerated.

As such, there is a constant demand for new therapeutic regimens for the treatment of mental illness and other psychiatric disorders.

### SUMMARY

Provided herein are methods for treating severe anxiety, major depressive disorder (MDD) single or recurrent, persistent depressive disorder (cyclothymia), treatment resistant depression (TRD), disruptive mood regulation disorder, bipolar 1 disorder, bipolar 2 disorder, premenstrual dysphoric disorder, schizoaffective disorder, adjustment disorder, complex regional pain syndrome (CRPS) type 1, CRPS type 2, chronic neuropathic pain, chronic pain syndromes, chronic low back pain, fibromyalgia, migraine headaches, chronic neuropathic pain, acute neuropathic pain, or irritable bowel syndrome, in a subject, comprising administering an effective amount of oxytocin, and an effective amount of a neuroplasticity agent, and optionally an effective amount of an anti-inflammatory agent to a subject in need thereof.

In some embodiments, the neuroplasticity agent is one or more of racemic ketamine, esketamine (Spravato), (R)-ketamine, (2R,6R)-hydroxynorketamine (HNK), psilocybin, 3,4-Methyl enedioxymethamphetamine (MDMA), N,N-dimethyltryptamine (DMT or N,N-DMT), lysergic acid diethylamide (LSD), dextromethorphan, nuedexta (a combination of dextromethorphan and quinidine), deudextromethorphan (AVP-786), axsome (AXS-05), dextromethadone (REL-1017), or zuranolone (SAGE-217).

Also provided herein is a method for treating post-partum depression or peripartum depression, in a subject, comprising administering an effective amount of oxytocin and an effective amount of a neuroplasticity agent to a subject in need thereof.

In some embodiments, the neuroplasticity agent is brexanolone or zuranolone (SAGE-217).

Also provided is a method for treating generalized anxiety disorder (GAD), panic disorder, post-traumatic stress disorder, separation anxiety disorder, obsessive compulsive disorder (OCD), social anxiety disorder, body dysmorphic disorder, anorexia nervosa, bulimia nervosa, binge eating disorder, in a subject, comprising administering an effective amount of oxytocin and an effective amount of a neuroplasticity agent to a subject in need thereof.

Also provided is a method for treating chronic fatigue syndrome in a subject, comprising administering an effective amount of oxytocin and an effective amount of a neuroplasticity agent to a subject in need thereof.

In some embodiments of the methods described herein, the treating comprises an acute, or stabilization phase, and a maintenance phase, where optionally the acute phase comprises administering a first dose of oxytocin prior to administering the neuroplasticity agent, optionally followed by an anti-inflammatory agent. In some embodiments, the oxytocin, neuroplasticity agent, and/or anti-inflammatory agent, are administered intranasally.

It is contemplated that the disclosed treatment allows a subject suffering from one or more of the aforementioned diseases or disorders to stimulate and regrow, or more quickly stimulate and regrow, neuronal connections in the brain with positive effects on mental and/or physical wellbeing.

### DETAILED DESCRIPTION

The following description sets forth exemplary embodiments of the present technology. It should be recognized, however, that such description is not intended as a limitation on the scope of the present disclosure but is instead provided as a description of exemplary embodiments.

Unless otherwise defined, all technical and scientific terms used herein have the same meaning as commonly understood by one of ordinary skill in the art to which this disclosure belongs.

The disclosures illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

The term “about” refers to a variation of ±1%, ±3%, ±5%, or ±10% of the value specified. For example, “about 50” can in some embodiments includes a range of from 45 to 55. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. Also, the singular forms “a” and “the” include plural references unless the context clearly dictates otherwise. Thus, e.g., reference to “a cannabinoid” includes a plurality of such compounds.

As used herein, “treatment” or “treating” is an approach for obtaining a beneficial or desired result, such as a clinical result. For purposes of this disclosure, beneficial or desired clinical results include, but are not limited to, alleviation of a symptom and/or diminishment of the extent of a symptom and/or preventing a worsening of a symptom associated with a disease or condition. For use herein, the beneficial or desired clinical results include, but are not limited to, alleviation of a symptom and/or diminishment of the extent of a symptom and/or preventing a worsening of a symptom associated with a given disease or disorder. Preferably, treatment of a subject using the methods described herein is accompanied by no or fewer side effects than are associated with currently available therapies for the disease or condition and/or disorder the quality of life of the subject, in particular, such as treatment resistant subjects, subjects who have severe side effects to common treatment medications, or those where a typical standard of care is ineffective or contraindicated. Typical standard of care can include medication, therapy, including physical or psychotherapy, or both.

As used herein, the term “effective amount” intends such amount of a compound or composition of the disclosure which in combination with its parameters of efficacy and toxicity, as well as based on the knowledge of the practicing specialist should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved.

The term “neuroplasticity agent” is intended to refer to a compound or substance that increase dendritic spine density, growth, or enhance morphology. In certain instances, the agent may confer changes in synaptic plasticity or dendrite morphology by providing increased expression of pre- or postsynaptic plasticity-related proteins, as well as the density and/or function of axo-spinous synapses. Studies in both humans and animal models have demonstrated abnormal dendritic spine architecture in several psychiatric disorders, including depression and other stress-related illnesses. The negative impact of stress on the density and organization of dendritic spines is also thought to contribute to the behavioral deficits caused by stress exposure.

Specific neuroplasticity agents are shown below in Table 1. Such agents are commercially available or can be prepared and formulated according to published methods.

TABLE 1

| Compound name | Structure |
|---|---|
| Ketamine (racemic) | 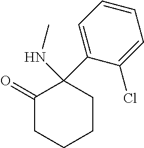 [SMILES: CNC1(c2ccccc2Cl)CCCCC1=O] |
| Esketamine | 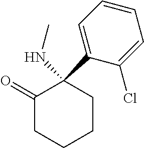 [SMILES: CN[C@]1(c2ccccc2Cl)CCCCC1=O] |
| (R)-ketamine | 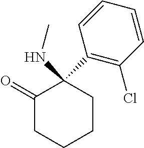 [SMILES: CN[C@@]1(c2ccccc2Cl)CCCCC1=O] |
| (2R,6R)- hydroxynorketamine (HNK) | 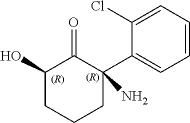 |
| Psilocybin | 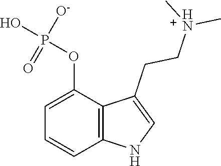 |
| 3,4-Methylenedioxy methamphetamine (MDMA) | 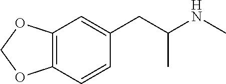 |
| N,N- Dimethyltryptamine (DMT or N,N-DMT) | 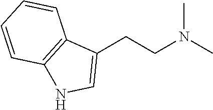 |
| Lysergic acid diethylamide (LSD) | 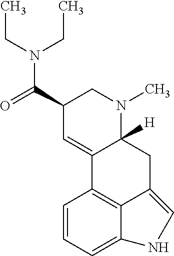 [SMILES: CCN(CC)C(=O)[C@@H]1C=C2c3cccc4[nH]cc(c34)C[C@H]2N(C)C1] |
| Dextromethorphan (DXM) | 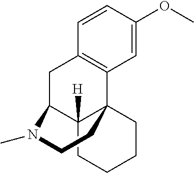 |
| Nuedexta | a combination of dextromethorphan and quinidine |
| deudextromethorphan | a combination of deudextromethorphan (d-DXM; |
| (AVP-786) | deuterated (d6) dextromethorphan (DXM)) and |
| | quinidine (Q) |
| axsome (AXS-05) | |
| dextromethadone (REL-1017) | 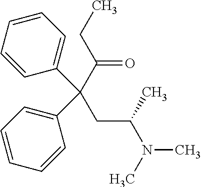 [SMILES: CCC(=O)C(C[C@H](C)N(C)C)(c1ccccc1)c1ccccc1] |
| zuranolone (SAGE- 217) | 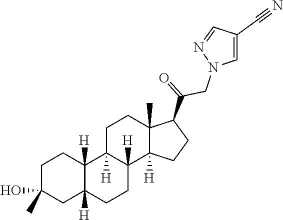 |
| Brexanolone | 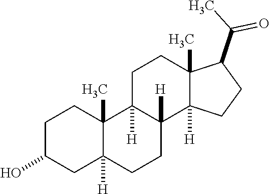 |

### Methods of Treatment

The neuropeptide oxytocin plays an evolutionarily conserved role in mammalian social behavior. Despite striking effects on animal social behavior after intracerebroventricular drug delivery, this delivery mode is impractical in humans. Intranasal oxytocin, which can be purchased from commercial sources, delivery provides a noninvasive alternative to increase central oxytocin activity, and has shown promise as a treatment for psychiatric illnesses. For example, research indicates that intranasal oxytocin administration improves theory of mind, memory for social cues, and increases gaze to the eye region. Intranasal oxytocin delivery is purported to increase central oxytocin concentrations via channels surrounding trigeminal and olfactory nerve fibers, which may facilitate increased activity at central oxytocin receptors. These findings, among others, have sparked interest in the use of intranasal oxytocin to treat psychiatric disorders characterized by social dysfunction; however, meta-analyses of oxytocin's effect in clinical populations have revealed mixed results to date.

Provided herein is a method for treating one or more disease or disorders selected from severe anxiety, major depressive disorder (MDD) single or recurrent, persistent depressive disorder (cyclothymia), treatment resistant depression (TRD), disruptive mood regulation disorder, bipolar 1 disorder, bipolar 2 disorder, premenstrual dysphoric disorder, schizoaffective disorder, adjustment disorder, complex regional pain syndrome (CRPS) type 1, CRPS type 2, chronic neuropathic pain, chronic pain syndromes, chronic low back pain, fibromyalgia, migraine headaches, chronic neuropathic pain, acute neuropathic pain, or irritable bowel syndrome, in a subject, comprising administering an effective amount of oxytocin and an effective amount of a neuroplasticity agent to a subject in need thereof.

Subjects in need of the methods described herein can be identified by standard methods. Pain is determined by a subject reporting of the same, whereas mental illnesses can be a more subjective diagnosis. A large number of psychiatric tests, scales, and forms have been created over the years to help in diagnosing mental illness and assisting in treatment and follow-up. These scales have demonstrated high levels of accuracy and validity and the results can give important clues to possible mental disorders that may warrant treatment via the methods described herein.

The Major Depression Inventory (MDI) is a brief, self-report mood questionnaire that allows clinicians to assess the presence of a depressive disorder according to DSM-IV. It is also used to assess the severity of depressive symptoms.

The mood disorder scale (MDQ), developed by Dr Robert M. A. Hirschfeld and colleagues, is a screening instrument for bipolar disorder. It includes 13 yes/no questions about bipolar symptoms and two additional questions about symptom co-occurrence and impaired functioning.

The Generalized Anxiety Disorder 7 item (GAD-7) was developed to diagnose generalized anxiety disorders and has been validated in 2740 primary-care patients. It has a sensitivity of 89% and a specificity of 82%. It is moderately good at screening 3 other common anxiety disorders: panic disorder (sensitivity 74%, specificity 81%), social anxiety disorder (sensitivity 72%, specificity 80%), and posttraumatic stress disorder (sensitivity 66%, specificity 81%).

The Clinical Global Impressions scale—CGI is a 3-item observer-rated scale commonly used to measure symptom severity, global improvement, and therapeutic response. Each component of the CGI is rated separately; it does not yield a global score. Items 1 and 2 are rated on a 7-point scale; item 3 is rated from 0 to 4 (when rating item 3, therapeutic efficacy and treatment-related adverse events should be taken into account).

The Hamilton Depression Rating Scale has proven useful for determining the level of depression before, during, and after treatment. It is based on the clinician's interview with the patient and probes symptoms such as depressed mood, guilty feelings, suicide, sleep disturbances, anxiety levels and weight loss. The interview and scoring takes about 15 minutes. The rater enters a number for each symptom construct that ranges from 0 (not present) to 4 (extreme symptoms).

The Bipolar Clinical Scale or Bipolar Spectrum Diagnostic Scale (BSDS) was developed by Ronald Pies, MD and was later refined and tested by S. Nassir Ghaemi, MD, MPH and colleagues. The BSDS arose from Pies's experience as a psychopharmacology consultant, where he was frequently called on to manage cases of “treatment-resistant depression.” In Pies's experience, most of these cases eventually proved to be undiagnosed bipolar spectrum disorder. The question items of the BSDS were based on those questions that Pies found most helpful in detecting not only severe cases of bipolar disorder but also patients who fall into the “softer” end of the bipolar spectrum (e.g., patients with a history of major depressive episodes and 1 or 2 episodes of elevated mood and energy that last only 1 to 3 days, thus not meeting DSM-IV criteria for hypomania). The BSDS was validated in its original version and demonstrated a high sensitivity (0.75 in bipolar I and 0.79 in bipolar II and not otherwise specified individuals). Its specificity was high (0.85), which confers a significant value to this diagnostic tool in the detection of a wide range of presentations within the bipolar spectrum. Ghaemi and colleagues determined that a score of 13 is the optimal threshold for specificity and sensitivity in the detection of bipolar spectrum disorders. The BSDS has two sections. The first part includes a series of 19 sentences that describe the main symptoms of bipolar spectrum disorders. Each sentence is linked to a blank space that should be checked by patients who decide that the statement is an accurate description of their feelings or behaviors. Each checked statement is assigned 1 point. The second portion of the BSDS asks the patient to select the degree to which the 19-item narrative “fits” his or her own experience. The scale offers four possibilities: “This story fits me very well, or almost perfectly” (6 points); “This story fits me fairly well” (4 points); “This story fits me to some degree, but not in most respects” (2 points); and “This story doesn't really describe me at all” (0 points).

The Hamilton Anxiety Scale (HAM-A) is a widely used interview scale that measures the severity of a patient's anxiety, based on 14 parameters, including anxious mood, tension, fears, insomnia, somatic complaints and behavior at the interview. Developed by M. Hamilton in 1959, the scale predates, of course, the current definition of generalized anxiety disorder (GAD). However, it covers many of the features of GAD and can be helpful also in assessing its severity. The major value of HAM-A is to document the results of pharmaco- or psychotherapy, rather than as a diagnostic or screening tool. It takes 15-20 minutes to complete the interview and scoring. Each item is simply given a 5-point score—0 (not present) to 4 (severe).

The Brief Psychiatric Rating Scale (BPRS) is a tool clinicians or researchers use to measure psychiatric symptoms such as anxiety, depression, and psychoses. Persons having or suspected of having schizophrenia or other psychotic disorder manifest the disorder in multiple ways. The BPRS assesses the level of 18 symptom constructs such as hostility, suspiciousness, hallucination, and grandiosity. It is particularly useful in gauging the efficacy of treatment in patients who have moderate to severe psychoses. It is based on the clinician's interview with the patient and observations of the patient's behavior over the previous 2-3 days. The patient's family can also provide the behavior report. The rater enters a number for each symptom construct that ranges from 1 (not present) to 7 (extremely severe). The time necessary to complete the interview and scoring can be as little as 20-30 minutes.

Persons taking any kind of antipsychotic medication need to be monitored for movement disorders. The AIMS (Abnormal Involuntary Movement Scale) aids in the early detection of tardive dyskinesia as well as providing a method for on-going surveillance. Although the incidence of TD has been relatively low in recent years, changes in prescribing may result in increased occurrence. Clinicians will need to be alert to these possibilities and employ tools that will help them pick up developing problems as soon as possible. This simple checklist takes only 10 minutes to complete and uses a 5-point rating scale for recording scores for 7 body areas: face, lips, jaw, tongue, upper extremities, lower extremities, and trunk.

The patient Health Questionnaire (PHQ-9) is the depression module, which scores each of the nine DSM-IV criteria as “0” (not at all) to “3” (nearly every day). It has been validated for use in primary care. It is generally not a screening tool for depression but it is used to monitor the severity of depression and response to treatment.

The Burns Anxiety Inventory (BAI) is an evaluation tool for measuring anxiety. The Burn Anxiety Inventory is a self-report anxiety assessment tool that evaluates anxious symptoms such as worry, nervousness or feelings of panic, or a racing heart. The higher the score, the greater the severity level of anxiety.

The Geriatric Depression Scale Short Form (GDS-S) is a 15-question scale developed as a basic screening measure for depression in older adults: a score of higher than 5 indicates that a more thorough clinical investigation is needed.

Studies have shown that inflammation in major depressive disorder (MDD) is a factor that should be evaluated and understood and it is related to the increase of proinflammatory cytokines such as interleukin 1-beta (IL-1β), interleukin 6 (IL-6), tumor necrosis factor-alpha (TNF-α), and interferon-gamma (IFN-γ), among others (Howren et al. Psychosom Med, 2009, 71, 171-186; Maes et al. Metab Brain Dis, 2009, 24, 27-53; Wong et al. Mol Psychiatry, 2008, 13, 800-812).

Reductions in brain-derived neurotrophic factor (BDNF), a protein of the neurotrophic family of growth factors, has been reported in different neuropsychiatric diseases. The BDNF blood levels are decreased in major depressive disorder subjects and increase after antidepressant treatment. See, Binder et al., Growth Factors, 2004, 22, 123-131; Ventriglia et al., BioMed Res Int, 2013, 2013, 901082; Pezet et al., Brain Res Rev, 2002, 40, 240-249; Björkholm et al., Neuropharmacology, 2016, 102, 72-79; Pandya et al., Asian J Psychiatr, 2013, 6, 22-28; Sen et al., Biol Psychiatry, 2008, 64, 527-532, Molendijk et al., Mol Psychiatry, 2014, 19, 791-800. Thus when the brain is subjected to chronic stress and anxiety, the number of synaptic connections in specific parts of the brain decrease. It is contemplated herein that administering a neuroplasticity agent can stimulate dendrite growth thereby restoring the lost synaptic connections, strengthen existing synaptic connections and even stimulate the growth of new neurons.

In some embodiments, the neuroplasticity agent is one or more of ketamine, including but not limited to, ketamine, esketamine, and (R)-ketamine, (2R,6R)-hydroxynorketamine (HNK), psilocybin, 3,4-Methylenedioxymethamphetamine (MDMA), N,N-dimethyltryptamine (DMT or N,N-DMT), lysergic acid diethylamide (LSD), dextromethorphan, nuedexta (a combination of dextromethorphan and quinidine), deudextromethorphan (AVP-786), axsome (AXS-05), dextromethadone (REL-1017), or zuranolone (SAGE-217).

Intravenous ketamine, for example, promotes new brain growth by increasing the number of synaptic connections in the brain. Chronic stress continues to stimulate the hypothalamic pituitary axis (HPA) which continues the release of inflammatory cytokines. The inflammatory cytokines gain access to the brain and cause reduced production and reduced reuptake of dopamine, norepinephrine, serotonin and BDNF. The reduced BDNF prevents the normal growth, repair and maintenance of the various neuron's synaptic connections. The dopamine neurons reduce the tonic release of dopamine leading to varying levels of anhedonia and depressed mood. With decreased production and reuptake of serotonin, the patients experience depressed mood. With decreased production and reuptake of norepinephrine, the patient experiences reduced focus and concentration. The most consequential effect of chronic stress is the reduction and reduced reuptake of dopamine which will be discussed further below.

By administering a neuroplasticity agent, the dendrites in the brain can regrow and lost connections can be restored. With IV ketamine alone, it has been determined that, under most circumstances, a series of infusions (e.g., six) may last anywhere from one month to several (e.g., 4-6) months. When the positive effects begin to wear off, a single booster infusion is often needed to extend the positive effects for another month or longer. It is contemplated by using the methods described herein, the positive effects can be extended or enhanced over ketamine alone. The method described will also provide quicker growth of the dendrites and their synaptic connections with other neurons.

In some embodiments, provided is a method for treating one or more disease or disorders selected from severe anxiety, major depressive disorder (MDD) single or recurrent, persistent depressive disorder (cyclothymia), treatment resistant depression (TRD), disruptive mood regulation disorder, bipolar 1 disorder, bipolar 2 disorder, premenstrual dysphoric disorder, schizoaffective disorder, adjustment disorder, complex regional pain syndrome (CRPS) type 1, CRPS type 2, chronic neuropathic pain, chronic pain syndromes, chronic low back pain, fibromyalgia, migraine headaches, chronic neuropathic pain, acute neuropathic pain, or irritable bowel syndrome, in a subject, comprising administering an effective amount of oxytocin, an effective amount of a neuroplasticity agent, and optionally an effective amount of an anti-inflammatory agent, to a subject in need thereof.

In some embodiments, provided is a method for treating one or more disease or disorders selected from severe anxiety, major depressive disorder (MDD) single or recurrent, persistent depressive disorder (cyclothymia), treatment resistant depression (TRD), disruptive mood regulation disorder, bipolar 1 disorder, bipolar 2 disorder, premenstrual dysphoric disorder, schizoaffective disorder, adjustment disorder, complex regional pain syndrome (CRPS) type 1, CRPS type 2, chronic neuropathic pain, chronic pain syndromes, chronic low back pain, fibromyalgia, migraine headaches, chronic neuropathic pain, acute neuropathic pain, or irritable bowel syndrome, in a subject, comprising administering an effective amount of oxytocin and an effective amount of a neuroplasticity agent, to a subject in need thereof.

In some embodiments, provided is a method for treating one of severe anxiety, major depressive disorder (MDD) single or recurrent, persistent depressive disorder (cyclothymia), treatment resistant depression (TRD), disruptive mood regulation disorder, bipolar 1 disorder, bipolar 2 disorder, premenstrual dysphoric disorder, schizoaffective disorder, adjustment disorder, complex regional pain syndrome (CRPS) type 1, CRPS type 2, chronic neuropathic pain, chronic pain syndromes, chronic low back pain, fibromyalgia, migraine headaches, chronic neuropathic pain, acute neuropathic pain, or irritable bowel syndrome, in a subject, comprising administering an effective amount of oxytocin and an effective amount of ketamine, including but not limited to, racemic ketamine (mixture of enantiomers), esketamine, or (R)-ketamine, to a subject in need thereof.

Also provided herein are methods for treating post-partum depression or peripartum depression, in a subject, comprising administering an effective amount of oxytocin, an effective amount of a neuroplasticity agent, and optionally an effective amount of an anti-inflammatory agent to a subject in need thereof.

In some embodiments, the neuroplasticity agent is brexanolone or zuranolone (SAGE-217).

Also provided herein are methods for treating generalized anxiety disorder (GAD), panic disorder, post-traumatic stress disorder, separation anxiety disorder, obsessive compulsive disorder (OCD), social anxiety disorder, body dysmorphic disorder, anorexia nervosa, bulimia nervosa, binge eating disorder, in a subject, comprising administering an effective amount of oxytocin, an effective amount of a neuroplasticity agent, and optionally an effective amount of an anti-inflammatory agent to a subject in need thereof.

Also provided is a method for treating chronic fatigue syndrome in a subject, comprising administering an effective amount of oxytocin, an effective amount of a neuroplasticity agent, and optionally an effective amount of an anti-inflammatory agent to a subject in need thereof.

In some embodiments, the subject is from a pre-adult or adolescent (e.g., from 16-18 years old). In some embodiments, the subject is an adult from 18-65 years old. In some embodiments, the subject is geriatric (e.g., older than 65, or from 66-75 years old). In some embodiments, the subject is male. In some embodiments, the subject is female.

In some embodiments, the oxytocin is administered intravenously. In some embodiments, the oxytocin is administered intranasally. In some embodiments, the neuroplasticity agent is administered intravenously. In some embodiments, the neuroplasticity agent is administered intranasally. In some embodiments, the anti-inflammatory agent is administered intravenously, intramuscularly or intranasal.

It has been noted that questions remain about the ideal outcome measures used in clinical trials to evaluate a subjects response to many of these tests. Many outcome measures currently in use, including those described above, were designed to measure change in symptoms that occur over weeks to months rather than hours to days. For instance, changes in insomnia or appetite cannot reasonably be assessed over a period of hours. Yet, most care providers (e.g., physicians or therapists) use these scales, suggesting that more accurate ways to capture the clinical effects of these agents are needed. It is possible that identifying unidimensional constructs that can help parse the heterogeneity of depressive symptoms will ultimately create more refined rating scale scores to more successfully illuminate connections between specific symptoms and underlying pathophysiology (Ioline et al., Current Status 1 CNS Drugs, 2021, 35(5), 527-543; Wilkinson et al., Drug Discov Today, 2019, 24, 606-615; Ballard et al., J Affect Disord, 2018, 231, 51-57.

Major depressive disorder is typically characterized by a depressed mood with loss of interest and pleasure in almost all activities for at least a two-week period.

In some embodiments, the subject exhibits one or more symptoms of depression, such as sleep disturbances, change in energy levels, or difficulty concentrating, anhedonic symptoms (inability to feel pleasure), dysphoric symptoms (state of unease or dissatisfaction), dissociative symptoms, an externalization of anger, or aggressive symptoms.

In some embodiments, the subject further suffers from one or more of obsessive-compulsive disorder, a trauma or stressor-related disorder, a dissociative disorder, acute stress disorder, adjustment disorder, disinhibited social engagement disorder, reactive attachment disorder, somatic symptom, a feeding or eating disorder, a sleep disorder, or a substance-related or addictive disorder.

In some embodiments, the disease or disorder is not a neuropsychiatric condition selected from the group consisting of post-traumatic stress disorder (PTSD), opioid use disorder (OUD), cocaine use disorder (CUD), unipolar post-partum depression (PPD), or combinations thereof.

A rating scale for determining severity of mental disease or disorder termed a “Reward/Enjoyment Scale”, including, but not limited to, major depressive disorder, is described herein. The Reward/Enjoyment Scale described herein (See Example 4) can be used to determine a treatment method as well as when to continue treating a subject in the acute, or stabilization phase vs when to transition to “chronic” treatments, i.e., the “maintenance” phase.

In some embodiments, the method comprises an acute “stabilization” phase and a “maintenance” phase.

### Acute “Stabilization” Phase

The acute “stabilizing” phase typically comprises a series of treatments wherein at each treatment the subject is administered oxytocin and a neuroplasticity agent, optionally followed by an anti-inflammatory agent, one or more times a week, such as 1, 2, or 3 times per week, for a period of 1-6 weeks, or 1-5 weeks, or 1-4 weeks, or 1-3 weeks, or 1-2 weeks, or 2-6 weeks, or 2-5 weeks, or 2-4 weeks, or 2-3 weeks, or 3-5 weeks, or 4-5 weeks, or 3-6 weeks, or 3-5 weeks, or 3-4 weeks, or 4-5 weeks, or 4-6 weeks, or 5-6 weeks, depending on the severity of the initial illness and/or the improvement of the illness. In certain embodiments, the acute “stabilizing” phase lasts about 4-5 treatments, or 2-3 weeks. Severity and improvement can be determined by clinical methods known in the art such as, but not limited to, those described herein. The more negative (−1 to −10 on a reward/enjoyment scale) a patient is, the more treatments it will take to get the patient into the positive zone. The amount of the patient's effort into applying positive coping skills (e.g., mindfulness, meditation, psychotherapy, proper diet, exercise and regulating sleep) will directly correlate with the overall antidepressant response.

The “negative” zone represents varying degrees of anhedonia as well as reward or relief from thinking, saying or doing negative things. A −1 is the least negative and a −10 is the most negative in the negative zone on the reward/enjoyment scale. When a patient loses synaptic connections in the reward pathways of the brain, the dopamine neurons seemingly go into an inactive state and the tonic release of dopamine decreases. When enough dopamine neurons go inactive, the low dopamine (hypodopaminergic) state will cause the patient to experience anhedonia, dysphoria, psychomotor slowing and a lack of motivation to seek out positive rewards. Those feelings will continue to worsen as the patient goes more negative (towards −10) in the negative zone.

Patients discover that they only get reward or relief from negative thoughts, statements or actions. They begin to seek out negative rewards to get relief from the dysphoria that they feel. The patients often develop a false narrative to support their negative thoughts, statements or actions. When their false narrative is challenged by others (typically loved ones), it can cause the patient to defend their false narrative and want to further isolate themselves from others. The negative zone has not yet been defined in the medical literature and has been a phenomena that has been observed.

As the patient loses more synaptic connections and they progress deeper into the negative zone, they seemingly get more reward and/or relief from their negative thoughts, statements and actions. Since the dopamine neurons are becoming less active as the patient continues to lose synaptic connections, it is postulated that the patients experience more relief through an up-regulation (or increased proliferation) of their mu opioid receptors. It is also possible that there is an up-regulation of their dopamine receptors (or increased proliferation) as the amount of dopamine released has been reduced. Thereby, as the patients go deeper into the negative zone, they get even more relief from things that cause more emotional and even physical pain. This theory would support why people deep in the negative zone experience immediate relief from non-suicidal self-injury (NSSI). The theory would also explain why people deeper in the negative zone get relief from suicidal thoughts. The thought of suicide causes them to feel like there will be an end to their pain and suffering, which makes them feel better.

It was hypothesized that the patient's own oxytocin was being released during the ketamine infusion, and this was likely a significant reason as to how ketamine was helping to relieve anxiety given its known calming effect on the limbic system. It was then contemplated that other rapid neuroplasticity agents which also stimulate the release of oxytocin (i.e., Psilocybin, MDMA) might play a role in neurogenesis and provide a reduction in anxiety levels. It was then observed that administering oxytocin prior to the ketamine treatment resulted in a much more calm and euphoric ketamine experience. The extra calming effect was greatly appreciated by patients suffering from anxiety disorders. It was then observed that patients were recovering much more quickly from depression and anxiety than just ketamine alone. It is imperative that patients get as quickly to the positive zone as possible. It had been seen that ketamine treatments alone were a lot slower to relieve the effects of anxiety than they were with depression. With the addition of oxytocin treatment, it was observed that patients were experiencing a much quicker recovery from anxiety than was witnessed from ketamine infusions alone. Anxiety is a major contributor to chronic inflammation, which decreases BDNF and the continued loss of synaptic connections. Reducing anxiety through the addition of exogenous oxytocin has significantly reduced the inflammation in the brain and thereby is contemplated to enhance the neuroplasticity effects from the ketamine.

Oxytocin enhances the limbic system and what is deemed as “safe” or “unsafe.” This applies to people, places and things. There are varying levels of “safe” and “unsafe.” When the limbic system detects a threat and oxytocin is present, the feeling of “unsafe” will be enhanced motivating the person to avoid, fight or get away from that person, place or thing. When the limbic system determines a person, place or thing is “safe” in the presence of oxytocin, the person will be motivated to be involved with that person, place or thing. When a person feels connected to someone and that person is safe, physical touch will enhance the release of additional oxytocin causing a soothing and calming feeling. The opposite can be true as well. When a person deemed “unsafe” touches an individual, more oxytocin is released and that person will feel an enhanced urge to fight or run away from that “unsafe” individual. When determining whether a patient should receive oxytocin prior to the neuroplasticity agent, the patient's level of reactivity should first be assessed by a physician. Oxytocin administration should be avoided when dealing with a highly reactive patient (i.e., acutely suicidal, homicidal).

The anti-inflammatory agent is used after the neuroplasticity agent to decrease the amount of inflammation present thereby allowing neurogenesis to proceed uninhibited. The neurogenesis is enhanced by reducing the effect that inflammation has on reducing the release of BDNF. The anti-inflammatory agent may cause an increased psychedelic effect when given with the ketamine. Thus, in some embodiments, the anti-inflammatory agent is administered after the psychedelic effects from the neuroplasticity agent have decreased.

In some embodiments, the acute “stabilization” phase comprises administering a first dose of oxytocin prior to administering the neuroplasticity agent. In some embodiments, the acute phase comprises intravenously administering a first dose of oxytocin prior to intravenously administering the neuroplasticity agent. In some embodiments, the acute phase comprises intranasally administering a first dose of oxytocin prior to intravenously administering the neuroplasticity agent. In some embodiments, the acute phase comprises intravenously administering a first dose of oxytocin prior to intranasally administering the neuroplasticity agent. In some embodiments, the acute phase comprises intranasally administering a first dose of oxytocin prior to intranasally administering the neuroplasticity agent.

In some embodiments, the acute phase comprises administering a first dose of the neuroplasticity agent prior to administering oxytocin. In some embodiments, the acute phase comprises intravenously administering a first dose of the neuroplasticity agent prior to intravenously administering oxytocin. In some embodiments, the acute phase comprises intranasally administering a first dose of the neuroplasticity agent prior to intravenously administering oxytocin. In some embodiments, the acute phase comprises intravenously administering a first dose of the neuroplasticity agent prior to intranasally administering oxytocin. In some embodiments, the acute phase comprises intranasally administering a first dose of the neuroplasticity agent prior to intranasally administering oxytocin.

In some embodiments, the dose of oxytocin administered at each treatment in the acute phase is from about 0.1 to about 100 International Units (IU). In some embodiments, the dose of oxytocin administered at each treatment in the acute phase is 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 International Units (IU). The oxytocin can be administered as a single administration (via IV or intranasal administration) or over multiple (i.e., two or more) administrations during each treatment.

In some embodiments, the neuroplasticity agent administered in the acute phase is ketamine. In some embodiments, the neuroplasticity agent administered in the acute phase is esketamine. In some embodiments, the neuroplasticity agent administered in the acute phase is (R)-ketamine.

In some embodiments, the neuroplasticity agent administered in the acute phase is (2R,6R)-hydroxynorketamine (HNK).

In some embodiments, the neuroplasticity agent administered in the acute phase is psilocybin.

In some embodiments, the neuroplasticity agent administered in the acute phase is 3,4-Methyl enedioxymethamphetamine (MDMA).

In some embodiments, the neuroplasticity agent administered in the acute phase is N,N-dimethyltryptamine (DMT or N,N-DMT).

In some embodiments, the neuroplasticity agent administered in the acute phase is lysergic acid diethylamide (LSD).

In some embodiments, the acute phase comprises administering dextromethorphan.

In some embodiments, the neuroplasticity agent administered in the acute phase is nuedexta (a combination of dextromethorphan and quinidine).

In some embodiments, the neuroplasticity agent administered in the acute phase is deudextromethorphan (AVP-786).

In some embodiments, the neuroplasticity agent administered in the acute phase is axsome (AXS-05).

In some embodiments, the neuroplasticity agent administered in the acute phase is dextromethadone (REL-1017).

In some embodiments, the neuroplasticity agent administered in the acute phase is zuranolone (SAGE-217).

In some embodiments, the neuroplasticity agent administered in the acute phase is brexanolone.

In some embodiments, the dose of neuroplasticity agent administered at each treatment in the acute phase is from about 0.1 to about 300 mg. In some embodiments, the dose of ketamine administered at each treatment in the acute phase is 0.1, 0.2, 0.3, 0.4, 0.5, 0.6, 0.7, 0.8, 0.9, 1, 2, 3, 4, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 mg.

In some embodiments, the neuroplasticity agent is ketamine (e.g., racemic ketamine, esketamine, or (R)-ketamine) and is administered intranasally at an initial dose of 0.1, 0.2, 0.3, 0.4, 0.5, 0.6, 0.7, 0.8, 0.9, 1, 2, 3, 4, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 mg, optionally with a second intranasal dose administered 10-15 minutes later at a dose of 0.1, 0.2, 0.3, 0.4, 0.5, 0.6, 0.7, 0.8, 0.9, 1, 2, 3, 4, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 mg.

In some embodiments, the acute “stabilization” phase comprises a treatment about twice a week, or 2-3 times per week, wherein each treatment is separated by about two to four days, or by 3 days, or about 72 hours, from the previous infusion.

During the acute “stabilization” phase, the oxytocin may or may not be administered at each treatment.

In some embodiments, the acute “stabilization” phase comprises administering neuroplasticity agent intravenously at a total dose of about 30-100 mg, or about 45-90 mg, or about 50-75 mg, or about 60 mg, optionally over about 45-60 minutes, or about 50 minutes.

In some embodiments, the acute “stabilization” phase comprises administering ketamine (e.g., racemic ketamine, esketamine, or (R)-ketamine) intravenously at a total dose of about 30-100 mg, or about 45-90 mg, or about 50-75 mg, or about 60 mg, optionally over about 45-60 minutes, or about 50 minutes.

One meta-analysis of nine randomized, placebo-controlled studies found that (R,S)-ketamine's antidepressant effects after a single administration typically begin 40 min post-infusion, peak approximately 24 h later, and lose superiority to placebo 10-12 days post-infusion. Other meta-analyses have corroborated these findings. See, Kishimoto et al., Psychol Med, 2016, 46(7), 1459-1472; Caddy et al., Ther Adv Psychopharmacol, 2014, 4(2), 75-99; McGirr et al., Psychol Med, 2015, 45(4), 693-704; Newport et al., Am J Psychiatry, 2015, 172(10), 950-966.

In some embodiments, the method comprises further administering an anti-inflammatory agent during the acute “stabilization” phase. The anti-inflammatory agent can be administered before, concurrently with, and/or after the neuroplasticity agent.

In some embodiments, the method comprises further administering a nonsteroidal anti-inflammatory drug (NSAID) during the acute “stabilization” phase. The nonsteroidal anti-inflammatory drug (NSAID) can be administered before, concurrently with, and/or after the neuroplasticity agent.

In some embodiments, the method comprises further administering acetaminophen during the acute “stabilization” phase. The acetaminophen can be administered before, concurrently with, and/or after the neuroplasticity agent.

In some embodiments, the anti-inflammatory agent, nonsteroidal anti-inflammatory drug (NSAID), or acetaminophen is administered orally. In some embodiments, the anti-inflammatory agent, nonsteroidal anti-inflammatory drug (NSAID), or acetaminophen is administered intravenously.

In some embodiments, the anti-inflammatory agent, nonsteroidal anti-inflammatory drug (NSAID), or acetaminophen is administered at a dose of about 2-5 g/day, or about 3-5 g/day, or about 3-4 g/day, or about 2 g/day, or about 3 g/day, or about 4 g/day, or about 5 g/day.

In some embodiments, the method comprises further administering a nonsteroidal anti-inflammatory drug (NSAID), such as ketorolac (sold under the brand names Toradol®, and Biorolac® among others). In certain embodiments, the ketorolac is administered intravenously. In certain embodiments, the ketorolac is administered intramuscularly. In certain embodiments, the ketorolac is administered intranasally. In some embodiments, the ketorolac is administered at a dose of about 10 mg/day, or about 15 mg/day, or about 20 mg/day, or about 25 mg/day, or about 30 mg/day, or about 35 mg/day, or about 40 mg/day, or about 15-30 mg/day.

In the acute phase, the patient may show an improved scoring on one or more tests, such as those described herein. In certain embodiments, the methods described herein result in a patient feeling significantly less depression and/or anxiety as assessed by the PHQ-9 and/or the Burns Anxiety Inventory score. With certain patients, although the Total Score may indicate a lessor effect, the patient often feels more resilient and is able to maintain some emotional consistency and is able to more effectively “bounce-back” after life stressor (e.g., a break-up, a move, a loss, job stress, etc.). For example, a patient who starts with a Burns Anxiety Inventory score of 51-99 (Extreme Anxiety or Panic) before treatment (day 0), and after treatment exhibits a Burns Anxiety Inventory score of 21-30 (Moderate Anxiety) will likely feel more resilient even if the patient still has moderate anxiety. Thus, the acute phase may begin once the patient feels sufficient or enhanced resiliency to stressor(s), rather an amelioration of symptoms, even if the score does not reflect the same.

In certain embodiments, the patient's PHQ-9 score improves by at least about 10%, at least about 40%, or at least about 50%, or at least about 60%, or at least about 70%, or at least about 80% as determined by the PHQ-9. In certain embodiments, the patient's PHQ-9 score improves by at least about 10%, at least about 40%, or at least about 50%, or at least about 60%, or at least about 70%, or at least about 80% as determined by the PHQ-9, after treatment 4.

In certain embodiments, the patient's Burns Anxiety Inventory score improves by at least about 20%, at least about 35%, or at least about 40%, or at least about 50%, or at least about 50%, or at least about 75%, or at least about 80% as determined by the Burns Anxiety Inventory. In certain embodiments, the patient's Burns Anxiety Inventory score improves by at least about 20%, at least about 35%, or at least about 40%, or at least about 50%, or at least about 50%, or at least about 75%, or at least about 80% as determined by the Burns Anxiety Inventory, after treatment 4.

Accordingly, in some embodiments, the method comprises only the acute “stabilization” phase. However, in some embodiments, the method further comprises a “maintenance” phase. Once the patient is determined to be sufficiently improved, e.g., in the positive zone, based on their self-rated scores and physician assessment, the maintenance phase can begin.

### Maintenance Phase

In some embodiments, the method comprises a maintenance phase comprising one or more additional treatments, where the oxytocin may or may not be administered at all or at each treatment. The treatment can comprise the same or different dose and/or route administration of oxytocin or neuroplasticity agent as was used in the acute phase. During the maintenance phase, each treatment dose of the maintenance phase decreases, remain unchanged, or increases from the previous treatment.

In some embodiments, the maintenance phase comprises administering oxytocin at one or more treatments at substantially the same dose as was administered in the acute phase.

In some embodiments, the maintenance phase comprises administering oxytocin at one or more treatments at a lower dose than was administered in the acute phase. In certain embodiments, the maintenance phase comprises administering oxytocin at one or more treatments at a dose which is 5%, or 10%, or 15%, or 20%, or 25%, or 30%, or 40%, or 45%, or 50% lower than the dose that was administered in the acute phase.

In some embodiments, the maintenance phase comprises administering oxytocin at one or more treatments at a higher dose as was administered in the acute phase. In certain embodiments, the maintenance phase comprises administering oxytocin at one or more treatments at a dose which is 5%, or 10%, or 15%, or 20%, or 25%, or 30%, or 40%, or 45%, or 50% higher than the dose that was administered in the acute phase.

In some embodiments, the dose of oxytocin administered during the maintenance phase decreases as compared to the start of the maintenance phase.

Typically, the maintenance phase comprises one or more treatments comprising administering a neuroplasticity agent, with or without oxytocin at any given treatment.

In some embodiments, the maintenance phase comprises administering neuroplasticity agent at one or more treatments at substantially the same dose as was administered in the acute phase. In some embodiments, the maintenance phase comprises administering neuroplasticity agent at one or more treatments at a lower dose than was administered in the acute phase. In some embodiments, the maintenance phase comprises administering neuroplasticity agent at one or more treatments at a higher dose as was administered in the acute phase.

In some embodiments, the maintenance phase comprises administering neuroplasticity agent at one or more treatments at a lower dose than was administered in the acute phase. In certain embodiments, the maintenance phase comprises administering neuroplasticity agent at one or more treatments at a dose which is 5%, or 10%, or 15%, or 20%, or 25%, or 30%, or 40%, or 45%, or 50% lower than the dose that was administered in the acute phase.

In some embodiments, the maintenance phase comprises administering neuroplasticity agent at one or more treatments at a lower dose than was administered in the acute phase. In certain embodiments, the maintenance phase comprises administering neuroplasticity agent at one or more treatments at a dose which is 5%, or 10%, or 15%, or 20%, or 25%, or 30%, or 40%, or 45%, or 50% higher than the dose that was administered in the acute phase.

In some embodiments, the dose of neuroplasticity agent administered during the maintenance phase decreases as compared to the start of the maintenance phase. In some embodiments, the dose decreases by about 5%, or 10%, or 15%, or 20%, or 25%, or 30%, or 40%, or 45%, or 50% over the maintenance phase.

In some embodiments of the maintenance phase, such as those where the time between treatments is extended (e.g., from every 2-4 weeks to every 2-6 months), the treatment dose may increase for either or both of the neuroplasticity agent and/or oxytocin. In some embodiments, each treatment dose of the maintenance phase increases 5 mg, 10 mg, or 15 mg based on the patient's response from the previous treatment.

In some embodiments, the method comprises further administering an anti-inflammatory agent during the maintenance phase. The anti-inflammatory agent can be administered before, concurrently with, and/or after the neuroplasticity agent and/or oxytocin.

In some embodiments, the method comprises further administering a nonsteroidal anti-inflammatory drug (NSAID) during the maintenance phase. The nonsteroidal anti-inflammatory drug (NSAID) can be administered before, concurrently with, and/or after the neuroplasticity agent and/or oxytocin.

In some embodiments, the method comprises further administering acetaminophen during the maintenance phase. The acetaminophen can be administered before, concurrently with, and/or after the neuroplasticity agent and/or oxytocin.

In some embodiments of the maintenance phase, an anti-inflammatory agent, nonsteroidal anti-inflammatory drug (NSAID), or acetaminophen is administered orally. In some embodiments of the maintenance phase, an anti-inflammatory agent, nonsteroidal anti-inflammatory drug (NSAID), or acetaminophen is administered intravenously.

In some embodiments of the maintenance phase, an anti-inflammatory agent, nonsteroidal anti-inflammatory drug (NSAID), or acetaminophen is administered at a dose of about 2-5 g/day, or about 3-5 g/day, or about 3-4 g/day, or about 2 g/day, or about 3 g/day, or about 4 g/day, or about 5 g/day.

In some embodiments of the maintenance phase, the method comprises further administering a nonsteroidal anti-inflammatory drug (NSAID), such as ketorolac (sold under the brand names Toradol®, and Biorolac® among others).

In certain embodiments of the maintenance phase, the ketorolac is administered intravenously. In certain embodiments of the maintenance phase, the ketorolac is administered intramuscularly. In certain embodiments, the ketorolac is administered intranasally.

In some embodiments of the maintenance phase, the ketorolac is administered at a dose of about 10 mg/day, or about 15 mg/day, or about 20 mg/day, or about 25 mg/day, or about 30 mg/day, or about 35 mg/day, or about 40 mg/day, or about 15-30 mg/day.

In some embodiments, the maintenance phase comprises one treatment per week for 2-4 weeks, then one treatment every other week, or one treatment every 2-4 weeks for the first 6 months.

In some embodiments, the subject is isolated from human interaction. In some embodiments, the method further comprises the use of one or more adjunct therapies along with the methods described herein. Examples of adjunct therapies include e.g., psychotherapy, participation in social support groups, and/or additional therapies with one or more additional pharmaceutical agents.

### Compositions

It should also be appreciated that a specific dosage and treatment regimen for any particular subject will depend upon a variety of factors, including the formulation, route of administration, activity of the neuroplasticity agent employed, the age, body weight, general health, sex, diet, time of administration, rate of excretion, and the judgment of the treating physician, and the severity of the particular disease being treated.

The oxytocin, anti-inflammatory, and neuroplasticity agents can be delivered separately or together by any suitable route, as pharmaceutical compositions which can comprise any number of excipients. Excipients that can be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003), the disclosure of which is incorporated herein by reference. In certain embodiments, administration of the oxytocin, anti-inflammatory and neuroplasticity agent are each independently suitable for intravenous, intramuscular, subcutaneous, parenteral, spinal, intravitreal, or epidermal administration (e.g., by injection or infusion). The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intravitreal, and intrastemal injection and infusion. Alternatively, administration of one or more of oxytocin, anti-inflammatory agent, and neuroplasticity agent can be independently via a non-parenteral route, such as a topical, epidermal or mucosal route of administration, e.g., intranasally, orally, vaginally, rectally, sublingually or topically.

For administration by inhalation or intranasal routes, the oxytocin, anti-inflammatory and neuroplasticity agent can each be independently delivered in the form of a solution, suspension, emulsion, or semisolid aerosol from pressurized packs, or a nebulizer, usually with the use of a propellant, e.g., halogenated carbons derived from methane and Ethan, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the compound and a suitable powder base such as lactose or starch.

Pharmaceutical compositions comprising oxytocin and neuroplasticity agent, optionally with an anti-inflammatory agent, can each independently be in the form of sterile aqueous solutions or dispersions. They can also be formulated in a microemulsion, liposome, or other ordered structure suitable to high drug concentration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01% to about ninety-nine percent of active ingredient, or from about 0.1% to about 70%, or from about 1% to about 30% of active ingredient in combination with a pharmaceutically acceptable carrier.

Dosage regimens are adjusted to provide the optimum desired response (e.g., a therapeutic response). For example, several divided doses can be administered over time or the dose can be proportionally reduced or increased as indicated by the exigencies of the therapeutic situation. It is especially advantageous to formulate parenteral compositions in dosage unit form for ease of administration and uniformity of dosage.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1

Treatment for adult male or female subjects ages 18 to 65 years old having been diagnosed with one or more of major depressive disorder (MDD) single or recurrent, persistent depressive disorder (cyclothymia), treatment resistant depression (TRD), disruptive mood regulation disorder, bipolar 1 disorder, bipolar 2 disorder, premenstrual dysphoric disorder, schizoaffective disorder, adjustment disorder, complex regional pain syndrome (CRPS) type 1, CRPS type 2, chronic neuropathic pain, chronic pain syndromes, chronic low back pain, fibromyalgia, migraine headaches, chronic neuropathic pain, acute neuropathic pain, or irritable bowel syndrome.

### Timing:

Acute (stabilizing) phase is 2 times a week for 3-5 weeks depending on the severity of the initial illness. The more negative (−1 to −10 on a reward/enjoyment scale) a patient is, the more treatments it will take to get the patient into the positive zone. The amount of the patient's effort into applying positive coping skills (e.g., mindfulness, meditation, psychotherapy, proper diet, exercise and regulating sleep) will directly correlate with the overall antidepressant response. Once the patient is clearly in the positive zone, based on their self-rated scores and physician assessment, the maintenance phase will begin.

Chronic (maintenance) phase begins after the acute (stabilization) phase and may initially be once a week for 2-4 weeks and then transition to every other week. Once the patient is maintaining their benefits, a treatment may be required once every 2-4 weeks for the first 6 months. The physician may alter the maintenance schedule to meet the patient's needs, but no more than one treatment per week. The physician may lengthen the treatment schedule based on the patient's ability to maintain their positive coping skills and engage in psychotherapy regularly (weekly is preferable). The physician may schedule a treatment off the normal schedule if the patient is anticipating a significant stressor or group of stressors.

Treatment regimens for the acute phase are as shown below in Tables 1a and 1b.

TABLE 1a

| | | Route of | | Route of |
|---|---|---|---|---|
| Regi- | First | Adminis- | | Adminis- |
| men | Drug | tration | Second Drug | tration |
| 1a | Oxytocin | intranasal | racemic ketamine | intravenous |
| 2a | Oxytocin | intravenous | racemic ketamine | intravenous |
| 3a | Oxytocin | intranasal | racemic ketamine | intranasal |
| 4a | Oxytocin | intravenous | racemic ketamine | intranasal |
| 5a | Oxytocin | intranasal | Esketamine | intravenous |
| 6a | Oxytocin | intravenous | Esketamine | intravenous |
| 7a | Oxytocin | intranasal | Esketamine | intranasal |
| 8a | Oxytocin | intravenous | Esketamine | intranasal |
| 9a | Oxytocin | intranasal | (R)-ketamine | intravenous |
| 10a | Oxytocin | intravenous | (R)-ketamine | intravenous |
| 11a | Oxytocin | intranasal | (R)-ketamine | intranasal |
| 12a | Oxytocin | intravenous | (R)-ketamine | intranasal |
| 13a | Oxytocin | intranasal | (2R,6R)- | intravenous |
| | | | hydroxynorketamine | |
| | | | [HNK] | |
| 14a | Oxytocin | intravenous | (2R,6R)- | intravenous |
| | | | hydroxynorketamine | |
| | | | [HNK] | |
| 15a | Oxytocin | intranasal | Psilocybin | intravenous |
| 16a | Oxytocin | intravenous | Psilocybin | intravenous |
| 17a | Oxytocin | intranasal | MDMA | intravenous |
| 18a | Oxytocin | intravenous | MDMA | intravenous |
| 19 | Oxytocin | intranasal | DMT | intravenous |
| 20a | Oxytocin | intravenous | DMT | intravenous |
| 21a | Oxytocin | intranasal | LSD | intravenous |
| 22a | Oxytocin | intravenous | LSD | intravenous |
| 23a | Oxytocin | intranasal | dextromethorphan | intravenous |
| 24a | Oxytocin | intravenous | dextromethorphan | intravenous |
| 25a | Oxytocin | intranasal | Nuedexta | intravenous |
| | | | [a combination of | |
| | | | dextromethorphan | |
| | | | and quinidine] | |
| 26a | Oxytocin | intravenous | Nuedexta | intravenous |
| | | | [a combination of | |
| | | | dextromethorphan | |
| | | | and quinidine] | |
| 27a | Oxytocin | intranasal | deudextromethorphan | intravenous |
| | | | [AVP-786] | |
| 28a | Oxytocin | intravenous | deudextromethorphan | intravenous |
| | | | [AVP-786] | |
| 29a | Oxytocin | intranasal | axsome [AXS-05] | intravenous |
| 30a | Oxytocin | intravenous | axsome [AXS-05] | intravenous |
| 31a | Oxytocin | intranasal | dextromethadone | intravenous |
| | | | [REL-1017] | |
| 32a | Oxytocin | intravenous | dextromethadone | intravenous |
| | | | [REL-1017] | |
| 33a | Oxytocin | intranasal | Zuranolone | intravenous |
| | | | (SAGE-217) | |
| 34a | Oxytocin | intravenous | Zuranolone | intravenous |
| | | | (SAGE-217) | |

TABLE 1b

| | | Route of | Second | Route of |
|---|---|---|---|---|
| Regi- | | Adminis- | | Adminis- |
| men | First Drug | tration | Drug | tration |
| 1b | racemic ketamine | intravenous | Oxytocin | intranasal |
| 2b | racemic ketamine | intravenous | Oxytocin | intravenous |
| 3b | racemic ketamine | intranasal | Oxytocin | intranasal |
| 4b | racemic ketamine | intranasal | Oxytocin | intravenous |
| 5b | Esketamine | intravenous | Oxytocin | intranasal |
| 6b | Esketamine | intravenous | Oxytocin | intravenous |
| 7b | Esketamine | intranasal | Oxytocin | intranasal |
| 8b | Esketamine | intranasal | Oxytocin | intravenous |
| 9b | (R)-ketamine | intravenous | Oxytocin | intranasal |
| 10b | (R)-ketamine | intravenous | Oxytocin | intravenous |
| 11b | (R)-ketamine | intranasal | Oxytocin | intranasal |
| 12b | (R)-ketamine | intranasal | Oxytocin | intravenous |
| 13b | (2R,6R)- | intravenous | Oxytocin | intranasal |
| | hydroxynorketamine | | | |
| | [HNK] | | | |
| 14b | (2R,6R)- | intravenous | Oxytocin | intravenous |
| | hydroxynorketamine | | | |
| | [HNK] | | | |
| 15b | Psilocybin | intravenous | Oxytocin | intranasal |
| 16b | Psilocybin | intravenous | Oxytocin | intravenous |
| 17b | MDMA | intravenous | Oxytocin | intranasal |
| 18b | MDMA | intravenous | Oxytocin | intravenous |
| 19b | DMT | intravenous | Oxytocin | intranasal |
| 20b | DMT | intravenous | Oxytocin | intravenous |
| 21b | LSD | intravenous | Oxytocin | intranasal |
| 22b | LSD | intravenous | Oxytocin | intravenous |
| 23b | dextromethorphan | intravenous | Oxytocin | intranasal |
| 24b | dextromethorphan | intravenous | Oxytocin | intravenous |
| 25b | Nuedexta | intravenous | Oxytocin | intranasal |
| | [a combination of | | | |
| | dextromethorphan | | | |
| | and quinidine] | | | |
| 26b | Nuedexta | intravenous | Oxytocin | intravenous |
| | [a combination of | | | |
| | dextromethorphan | | | |
| | and quinidine] | | | |
| 27b | deudextromethorphan | intravenous | Oxytocin | intranasal |
| | [AVP-786] | | | |
| 28b | deudextromethorphan | intravenous | Oxytocin | intravenous |
| | [AVP-786] | | | |
| 29b | axsome [AXS-05] | intravenous | Oxytocin | intranasal |
| 30b | axsome [AXS-05] | intravenous | Oxytocin | intravenous |
| 31b | dextromethadone | intravenous | Oxytocin | intranasal |
| | [REL-1017] | | | |
| 32b | dextromethadone | intravenous | Oxytocin | intravenous |
| | [REL-1017] | | | |
| 33b | Zuranolone | intravenous | Oxytocin | intranasal |
| | (SAGE-217) | | | |
| 34b | Zuranolone | intravenous | Oxytocin | intravenous |
| | (SAGE-217) | | | |

### Dose:

Intranasal Oxytocin will be given primarily before the second medication regardless of whether the second medication is intravenous or inhalational. The oxytocin may be administered intravenously prior to or after the second medication. The dose of oxytocin will be: 0.1, 0.2, 0.3, 0.4, 0.5, 0.6, 0.7, 0.8, 0.9, 1, 2, up to 100 International Units (IU).

Intravenous (IV) Ketamine will be administered initially at a very low dose with the first treatment. 60 mg of ketamine will be diluted in a 60 mL syringe containing normal saline. The 60 ml syringe will be attached to a syringe pump with microtubing extending to the patient's IV. The initial rate will be set at 10-20 mL/hr depending on the indication for treatment. All subjects with anxiety based disorders should be started at 10 mL/hr. Every 8-10 minutes, the physician may increase the dose a small amount based on the patient's tolerance to the experience. A typical dose increase will be 5-10 mL/hr every 8-10 minutes until 50 minutes.

The patient will be given at least 10-15 minutes or longer as needed to recover. Their post treatment vitals should return to within 25% of the patient's pretreatment vitals. The post treatment vitals (heart rate, blood pressure, pulse oximetry, respiratory rate) should be within 25% of the pretreatment vitals before the patient monitors are removed. The only exception to remove the monitors sooner would be if the patient has to use the restroom before the vitals return to within 25% of the patient's pretreatment values. The patient should be transported to the restroom via wheelchair and assisted as needed to avoid any potential fall. The patient should be reattached to the patient monitors and meet the proper discharge criteria before they are allowed to leave the clinic. The patient will need to be driven home after the treatment by a responsible adult. The patient will not be allowed to drive, participate in any hazardous activities or sign legal documents for 24 hours after the treatment.

The ketamine dose at every subsequent appointment will either decrease, remain unchanged or be increased from the previous treatment dose. A typical increase would be 5 mg, 10 mg or 15 mg based on the patient's response from the previous treatment. Once the patient has achieved their optimal benefits, the dose of ketamine should remain the same or be slightly decreased based on the patient's comfort with the treatment dose.

Intranasal Ketamine: Initial dose will be either 0.1, 0.2, 0.3, 0.4, 0.5, 0.6, 0.7, 0.8, 0.9, 1, 2, up to 300 mg.

Second intranasal ketamine dose administered 10-15 minutes later based on patient's tolerance (physician determined). It will be either 0.1, 0.2, 0.3, 0.4, 0.5, 0.6, 0.7, 0.8, 0.9, 1, 2, up to 300 mg.

The first intranasal dose of the ketamine treatment will be at a very low introductory dose to determine a patient's sensitivity to the medication. The second dose will be higher based on the patient's response to the first dose. A third low dose may be given only for the first treatment.

After the initial treatment, only 2 doses of intranasal Ketamine will be given separated by 10-15 minutes as determined by the supervising physician. The oxytocin dose will remain constant throughout the treatment process. The doses will go up with each treatment based on the patient's overall response and tolerance of the treatment experience.

Once in the maintenance phase, the patient may come in to the treatment clinic for a weekly oxytocin intranasal treatment.

Doses of other medications listed above combined with Oxytocin will be determined by future research studies.

### Example 2

Treatment for adult female subjects ages 18 to 65 years old having been diagnosed with one or more of post-partum depression or peripartum depression.

### Timing:

Acute (stabilizing) phase is 2 times a week for 3-5 weeks depending on the severity of the initial illness. The more negative (−1 to −10 on a reward/enjoyment scale) a patient is, the more treatments it will take to get the patient into the positive zone. The amount of the patient's effort into applying positive coping skills (e.g., mindfulness, meditation, psychotherapy, proper diet, exercise and regulating sleep) will directly correlate with the overall antidepressant response. Once the patient is clearly in the positive zone, based on their self-rated scores and physician assessment, the maintenance phase will begin.

Chronic (maintenance) phase begins after the acute phase and may initially be once a week for 2-4 weeks and then transition to every other week. Once the patient is maintaining their benefits, a treatment may be required once every 2-4 weeks for the first 6 months. The physician may alter the maintenance schedule to meet the patient's needs, but no more than one treatment per week. The physician may lengthen the treatment schedule based on the patient's ability to maintain their positive coping skills and engage in psychotherapy regularly (weekly is preferable). The physician may schedule a treatment off the normal schedule if the patient is anticipating a significant stressor or group of stressors.

Treatment regimens for the acute phase are as shown below in Tables 1c and 1d.

TABLE 1c

| | | Route of | | Route of |
|---|---|---|---|---|
| Regimen | First Drug | Administration | Second Drug | Administration |
| 1c | Oxytocin | intranasal | Brexanolone | intravenous |
| 2c | Oxytocin | intravenous | Brexanolone | intravenous |
| 3c | Oxytocin | intranasal | Zuranolone | intravenous |
| | | | (SAGE-217) | |
| 4c | Oxytocin | intravenous | Zuranolone | intravenous |
| | | | (SAGE-217) | |

TABLE 1d

| | | Route of | | Route of |
|---|---|---|---|---|
| Regimen | First Drug | Administration | Second Drug | Administration |
| 1d | Brexanolone | intravenous | Oxytocin | intranasal |
| 2d | Brexanolone | intravenous | Oxytocin | intravenous |
| 3d | Zuranolone | intravenous | Oxytocin | intranasal |
| | (SAGE-217) | | | |
| 4d | Zuranolone | intravenous | Oxytocin | intravenous |
| | (SAGE-217) | | | |

### Dose:

Intranasal Oxytocin will be given primarily before the second medication regardless of whether the second medication is intravenous or inhalational. The oxytocin may be administered intravenously prior to or after the second medication. The dose of oxytocin will be: 0.1, 0.2, 0.3, 0.4, 0.5, 0.6, 0.7, 0.8, 0.9, 1, 2, up to 100 International Units (IU).

Intravenous (IV) Ketamine will be administered initially at a very low dose with the first treatment. 60 mg of ketamine will be diluted in a 60 mL syringe containing normal saline. The 60 ml syringe will be attached to a syringe pump with microtubing extending to the patient's IV. The initial rate will be set at 10-20 mL/hr depending on the indication for treatment. All subjects with anxiety based disorders should be started at 10 mL/hr. Every 8-10 minutes, the physician may increase the dose a small amount based on the patient's tolerance to the experience. A typical dose increase will be 5-10 mL/hr every 8-10 minutes until 50 minutes.

The patient will be given at 10-15 minutes or longer as needed to recover. Their post treatment vitals should return to within 25% of the patient's pretreatment vitals. The post treatment vitals (heart rate, blood pressure, pulse oximetry, respiratory rate) should be within 25% of the pretreatment vitals before the patient monitors are removed. The only exception to remove the monitors sooner would be if the patient has to use the restroom before the vitals return to within 25% of the patient's pretreatment values. The patient should be transported to the restroom via wheelchair and assisted as needed to avoid any potential fall. The patient should be reattached to the patient monitors and meet the proper discharge criteria before they are allowed to leave the clinic. The patient will need to be driven home after the treatment by a responsible adult. The patient will not be allowed to drive, participate in any hazardous activities or sign legal documents for 24 hours after the treatment.

The ketamine dose at every subsequent appointment will either decrease, remain unchanged or be increased from the previous treatment dose. A typical increase would be 5 mg, 10 mg or 15 mg based on the patient's response from the previous treatment. Once the patient has achieved their optimal benefits, the dose of ketamine should remain the same or be slightly decreased based on the patient's comfort with the treatment dose.

Intranasal Ketamine: Initial dose will be either 0.1, 0.2, 0.3, 0.4, 0.5, 0.6, 0.7, 0.8, 0.9, 1, 2, up to 300 mg.

Second intranasal ketamine dose administered 10-15 minutes later based on patient's tolerance (physician determined). It will be either 0.1, 0.2, 0.3, 0.4, 0.5, 0.6, 0.7, 0.8, 0.9, 1, 2, up to 300 mg.

The first intranasal dose of the ketamine treatment will be at a very low introductory dose to determine a patient's sensitivity to the medication. The second dose will be higher based on the patient's response to the first dose. A third low dose may be given only for the first treatment.

After the initial treatment, only 2 doses of intranasal Ketamine will be given separated by 10-15 minutes as determined by the supervising physician. The oxytocin dose will remain constant throughout the treatment process. The doses will go up with each treatment based on the patient's overall response and tolerance of the treatment experience.

Once in the maintenance phase, the patient may come in to the treatment clinic for a weekly oxytocin intranasal treatment.

Doses of other medications listed above combined with Oxytocin will be determined by future research studies.

### Example 3

Treatment for male or female subjects having been diagnosed with one or more of generalized anxiety disorder (GAD), panic disorder, post-traumatic stress disorder (PTSD), separation anxiety disorder, obsessive compulsive disorder (OCD), social anxiety disorder, body dysmorphic disorder, anorexia nervosa, bulimia nervosa, binge eating disorder, or chronic fatigue syndrome.

Treatment: see Examples 1 and 2.

Route: see Examples 1 and 2.

Timing: see Examples 1 and 2.

For pre-adult subjects (under 18 years, e.g., 16-18 years old) or geriatric (greater than 65 years, e.g., 66-75 years old)

Dose: The initial dose for these disorders will be much smaller. There will be a much slower progression of the dose based on the patient's comfort level.

For pre-adult subjects, the treatment will be at lower initial doses of Neuroplasticity Medication than major depressive disorder (MDD).

For geriatric subjects, the treatment will be at lower doses of neuroplasticity agent.

### Example 4

The following Example shows treatment results for several patients. Table 2 shows patient information and diagnosis at the start of treatment.

TABLE 2

| Patient No. | Age/Sex | Diagnosis |
|---|---|---|
| 1 | 31/F | Depression, Anxiety, and PTSD |
| 2 | 33/M | Anxiety, Depression, and OCD |
| 3 | 55/F | Bipolar 1, Alcohol use Disorder, and |
| | | Depression |
| 4 | 15/M | OCD and Depression |
| 5 | 21/F | Bipolar 1 and Schizoaffective Disorder |
| 6 | 29/F | Depression and Anxiety |
| 10 | 41/F | PTSD, Depression, and Anxiety |
| 11 | 30/F | Depression, ADHD, and Anxiety |
| 14 | 28/M | Depression and Gambling Addiction |
| 15 | 44/M | Depression and Anxiety |
| 16 | 34/F | Depression, Anxiety, and Chronic Pain |
| 18 | 22/M | Depression, Anxiety, and Borderline |
| | | Personality Disorder |
| 19 | 69/F | Depression and Anxiety |

Results below were determined using the Patient Health Questionnaire (PHQ-9) and Burn Anxiety Inventory. In the following, the dose for the Burns Anxiety Inventory was the same dose as presented in the PHQ-9 data tables. In the PHQ-9 data in the tables, severity is reported as: EXT or ED (Extremely Difficult), VD (Very Difficult), SD (Somewhat difficult), and ND (Not Difficult). The PHQ-9 questionnaire was modified only to reflect the shortened time period between treatments in order to assess improvement during those time periods.

Patient 1: Patient has had four different suicide attempts and four involuntary hospitalizations. Patient tried over 15 antidepressants without relief. Patient presented with severe suicidality, depression, and anxiety. Current medications: Mirtazapine 15 mg once a day; propranolol 10 mg as needed; oral contraceptive. The patient entered the positive zone after treatment 3. The patient had never experienced relief so fast. The patient experienced some difficult stressors during the maintenance phase and recovered well. The patient feels very stable.

PHQ-9 DEPRESSION DATA

| | | | | DOSE PER TREATMENT | | |
|---|---|---|---|---|---|---|
| | | TOTAL | | KETAMINE | OXYTOCIN | TORADOL |
| Treatment | Day | SCORE (0-27) | SEVERITY | (mg/kg) | (IU) | (mg) |
| 1 | 0 | 27 | ED | 0.69 | 24 | 0 |
| 2 | 2 | 24 | ED | 0.96 | 24 | 30 |
| 3 | 6 | 11 | ND | 1.04 | 24 | 30 |
| 4 | 9 | 4 | ND | 1.04 | 24 | 30 |
| 5 | 12 | 2 | ND | 1.04 | 24 | 30 |
| 6 | 19 | 3 | ND | 1.13 | 24 | 30 |
| 7 | 34 | 2 | ND | 1.13 | 24 | 30 |
| 8 | 44 | 0 | ND | 1.13 | 24 | 30 |
| 9 | 57 | 21 | ED | 1.13 | 24 | 30 |
| 10 | 62 | 13 | SD | 1.13 | 24 | 30 |
| 11 | 69 | 16 | VD | 1.13 | 24 | 30 |
| 12 | 72 | 19 | VD | 1.13 | 24 | 30 |
| 13 | 75 | 12 | SD | 1.13 | 24 | 30 |
| 14 | 79 | 8 | SD | 1.13 | 24 | 30 |
| 15 | 85 | 5 | SD | 1.22 | 24 | 30 |
| 16 | 91 | 4 | ND | 1.22 | 24 | 30 |
| 17 | 104 | 9 | SD | 1.22 | 24 | 30 |
| 18 | 120 | 3 | ND | 1.22 | 24 | 30 |
| 19 | 159 | 1 | ND | 1.22 | 24 | 30 |
| 20 | 191 | 1 | ND | 1.22 | 24 | 30 |

BURNS ANXIETY INVENTORY DATA

| | | | | ANXIOUS | ANXIOUS | PHYSICAL |
|---|---|---|---|---|---|---|
| | | TOTAL | | FEELINGS | THOUGHTS | SYMPTOMS |
| Treatment | Day | SCORE (0-99) | SEVERITY | (0-18) | (0-33) | (0-48) |
| 1 | 0 | 84 | EXTREME | 16 | 27 | 41 |
| 2 | 2 | 76 | EXTREME | 17 | 27 | 32 |
| 3 | 6 | 21 | MODERATE | 4 | 4 | 13 |
| 4 | 9 | 10 | BORDERLINE | 3 | 3 | 4 |
| 5 | 12 | 10 | BORDERLINE | 2 | 3 | 5 |
| 6 | 19 | 7 | BORDERLINE | 3 | 0 | 4 |
| 7 | 34 | 11 | MILD | 3 | 4 | 4 |
| 8 | 44 | 5 | BORDERLINE | 2 | 0 | 3 |
| 9 | 57 | 70 | EXTREME | 13 | 27 | 30 |
| 10 | 62 | 33 | SEVERE | 7 | 12 | 14 |
| 11 | 69 | 70 | EXT | 14 | 24 | 32 |
| 12 | 72 | 59 | EXT | 13 | 25 | 21 |
| 13 | 75 | 26 | MODERATE | 5 | 12 | 9 |
| 14 | 79 | 27 | MODERATE | 4 | 10 | 13 |
| 15 | 85 | 12 | MILD | 3 | 5 | 4 |
| 16 | 91 | 20 | MILD | 2 | 8 | 10 |
| 17 | 104 | 39 | SEVERE | 9 | 14 | 16 |
| 18 | 120 | 12 | MILD | 2 | 6 | 4 |
| 19 | 159 | 10 | BORDERLINE | 3 | 4 | 3 |
| 20 | 191 | 6 | BORDERLINE | 1 | 3 | 2 |

Patient 2: Patient had been on sertraline 200 mg once a day for 4 years. The patient was able to wean off of it during a stress-free period approx. 1 year before present treatment. With stress, patient's OCD returned and was worse than previously experienced. The patient restarted sertraline after treatment 4. The patient entered into the positive zone after the treatment 6. Treatments 10-12 were intranasal ketamine. Patient did not go above 100 mg of sertraline and was more stable after last treatment.

PHQ-9 DEPRESSION DATA

| | | | | DOSE PER TREATMENT | | |
|---|---|---|---|---|---|---|
| | | TOTAL | | KETAMINE | OXYTOCIN | TORADOL |
| Treatment | Day | SCORE (0-27) | SEVERITY | (mg/kg) | (IU) | (mg) |
| 1 | 0 | 17 | VD | 0.39 | 24 | 30 |
| 2 | 4 | 13 | SD | 0.54 | 24 | 30 |
| 3 | 7 | 14 | SD | 0.70 | 24 | 30 |
| 4 | 12 | 8 | VD | 0.86 | 24 | 30 |
| 5 | 15 | 11 | VD | 1.02 | 24 | 30 |
| 6 | 18 | 11 | VD | 1.18 | 24 | 30 |
| 7 | 21 | 8 | SD | 1.29 | 24 | 30 |
| 8 | 26 | 7 | VD | 1.40 | 24 | 30 |
| 9 | 29 | 7 | VD | 1.40 | 24 | 30 |
| 10 | 33 | 7 | VD | 1.61 | 24 | 30 |
| 11 | 36 | 8 | VD | 1.61 | 24 | 30 |
| 12 | 48 | 5 | SD | 1.61 | 24 | 30 |
| 13 | 69 | 3 | SD | 1.61 | 24 | 30 |

BURNS ANXIETY INVENTORY DATA

| | | | | ANXIOUS | ANXIOUS | PHYSICAL |
|---|---|---|---|---|---|---|
| | | TOTAL | | FEELINGS | THOUGHTS | SYMPTOMS |
| Treatment | Day | SCORE (0-99) | SEVERITY | (0-18) | (0-33) | (0-48) |
| 1 | 0 | 47 | SEVERE | 14 | 11 | 22 |
| 2 | 4 | 36 | MODE | 13 | 11 | 12 |
| 3 | 7 | 31 | SEVERE | 11 | 10 | 10 |
| 4 | 12 | 31 | SEVERE | 13 | 8 | 10 |
| 5 | 15 | 33 | SEVERE | 10 | 13 | 10 |
| 6 | 18 | 33 | SEVERE | 9 | 14 | 10 |
| 7 | 21 | 31 | SEVERE | 9 | 12 | 10 |
| 8 | 26 | 33 | SEVERE | 11 | 12 | 10 |
| 9 | 29 | 28 | MODERATE | 8 | 9 | 11 |
| 10 | 33 | 28 | MODERATE | 10 | 11 | 7 |
| 11 | 36 | 29 | MODERATE | 8 | 11 | 10 |
| 12 | 48 | 28 | MODERATE | 11 | 12 | 5 |
| 13 | 69 | 20 | MILD | 9 | 6 | 6 |

Patient 3: Patient had been an active alcoholic since 18 years of age. Patient had been on several antidepressants before without success. Meds: Seroquel 100 mg at night; Klonopin 1 mg at night; Latuda 50 mg once daily. The patient entered the positive zone after treatment 4. The patient then started oxytocin treatment. The patient attributes the addition of the oxytocin for helping to stay sober. The patient continued to stay sober for 18 months. The patient weaned off medications and is only taking 50-100 mg of Seroquel at night.

PHQ-9 DEPRESSION DATA

| | | | | DOSE PER TREATMENT | | |
|---|---|---|---|---|---|---|
| | | TOTAL | | KETAMINE | OXYTOCIN | TORADOL |
| Treatment | Day | SCORE (0-27) | SEVERITY | (mg/kg) | (IU) | (mg) |
| 1 | 0 | 22 | ED | 0.52 | 0 | 0 |
| 2 | 2 | 22 | ED | 0.67 | 0 | 0 |
| 3 | 5 | 6 | ND | 0.76 | 0 | 0 |
| 4 | 8 | 7 | SD | 0.84 | 0 | 0 |
| 5 | 12 | 1 | SD | 0.84 | 0 | 0 |
| 6 | 15 | 0 | ND | 0.00 | 0 | 0 |
| 7 | 22 | 1 | ND | 0.00 | 0 | 0 |
| 8 | 47 | 1 | SD | 0.93 | 0 | 0 |
| 9 | 70 | 6 | VD | 0.93 | 0 | 0 |
| 10 | 91 | 2 | SD | 1.01 | 0 | 0 |
| 11 | 120 | 2 | ND | 1.01 | 0 | 0 |
| 12 | 148 | 0 | ND | 1.01 | 0 | 0 |
| 13 | 182 | 5 | SD | 1.01 | 24 | 0 |
| 14 | 217 | 2 | SD | 0.93 | 24 | 0 |
| 15 | 238 | 9 | SD | 0.00 | 24 | 0 |
| 16 | 252 | 3 | SD | 0.93 | 24 | 0 |
| 17 | 267 | 10 | VD | 0.00 | 24 | 0 |
| 18 | 280 | 11 | VD | 0.93 | 24 | 0 |
| 19 | 308 | 12 | VD | 0.93 | 24 | 0 |
| 20 | 329 | 5 | SD | 0.93 | 24 | 30 |
| 21 | 334 | 8 | SD | 0.00 | 0 | 0 |
| 22 | 357 | 4 | SD | 0.93 | 24 | 30 |
| 23 | 386 | 4 | SD | 0.93 | 24 | 30 |
| 24 | 399 | 3 | SD | 0.00 | 24 | 0 |
| 25 | 407 | 1 | SD | 0.00 | 24 | 0 |
| 26 | 414 | 4 | SD | 0.93 | 24 | 30 |
| 27 | 421 | 2 | SD | 0.00 | 24 | 0 |
| 28 | 432 | 3 | SD | 0.00 | 24 | 0 |
| 29 | 442 | 5 | SD | 0.93 | 24 | 30 |
| 30 | 470 | 3 | SD | 0.93 | 24 | 30 |
| 31 | 498 | 4 | SD | 0.93 | 24 | 30 |

BURNS ANXIETY INVENTORY DATA

| | | | | ANXIOUS | ANXIOUS | PHYSICAL |
|---|---|---|---|---|---|---|
| | | TOTAL | | FEELINGS | THOUGHTS (0- | SYMPTOMS |
| Treatment | Day | SCORE (0-99) | SEVERITY | (0-18) | 33) | (0-48) |
| 1 | 0 | 33 | SEVERE | 6 | 13 | 14 |
| 2 | 2 | 33 | SEVERE | 7 | 11 | 14 |
| 3 | 5 | 10 | BORDERLINE | 3 | 1 | 6 |
| 4 | 8 | 13 | BORDERLINE | 2 | 5 | 6 |
| 5 | 12 | 2 | MINIMAL | 1 | 0 | 1 |
| 6 | 15 | 0 | MINIMAL | 0 | 0 | 0 |
| 7 | 22 | 1 | MINIMAL | 0 | 0 | 1 |
| 8 | 47 | 1 | MINIMAL | 0 | 0 | 1 |
| 9 | 70 | 7 | BORDERLINE | 0 | 3 | 4 |
| 10 | 91 | 1 | MINIMAL | 0 | 0 | 1 |
| 11 | 120 | 1 | MINIMAL | 0 | 0 | 1 |
| 12 | 148 | 0 | MINIMAL | 0 | 0 | 0 |
| 13 | 182 | 6 | BORDERLINE | 0 | 2 | 4 |
| 14 | 217 | 0 | MINIMAL | 0 | 0 | 0 |
| 15 | 238 | 6 | BORDERLINE | 1 | 3 | 3 |
| 16 | 252 | 7 | BORDERLINE | 1 | 3 | 3 |
| 17 | 267 | 5 | BORDERLINE | 0 | 2 | 3 |
| 18 | 280 | 15 | MILD | 4 | 7 | 4 |
| 19 | 308 | 9 | BORDERLINE | 3 | 3 | 3 |
| 20 | 329 | 3 | MINIMAL | 0 | 0 | 3 |
| 21 | 334 | 2 | MINIMAL | 0 | 0 | 2 |
| 22 | 357 | 1 | MINIMAL | 0 | 0 | 1 |
| 23 | 386 | 2 | MINIMAL | 0 | 0 | 2 |
| 24 | 399 | 8 | BORDERLINE | 2 | 0 | 6 |
| 25 | 407 | 1 | MINIMAL | 0 | 0 | 1 |
| 26 | 414 | 2 | MINIMAL | 0 | 0 | 2 |
| 27 | 421 | 2 | MINIMAL | 0 | 0 | 2 |
| 28 | 432 | 7 | BORDERLINE | 0 | 1 | 6 |
| 29 | 442 | 1 | MINIMAL | 0 | 0 | 1 |
| 30 | 470 | 2 | MINIMAL | 0 | 0 | 2 |
| 31 | 498 | 3 | MINIMAL | 0 | 0 | 3 |

Patient 4: The patient couldn't tolerate any antidepressants (tried Zoloft without success, and had been taking 20 mg of Lexapro for 1 year at the beginning of treatment). The patient entered the positive zone after treatment 5. All treatments were with intranasal ketamine and intramuscular Toradol. It was observed that higher doses were necessary since some of the ketamine dripped down the back of the throat or out the nose. Once stabilized, the patient was doing great emotionally, socially and ended up earning straight A's by the end of the school year. The patient was able to give a public talk about how ketamine saved his life and how he no longer feels suicidal.

PHQ-9 DEPRESSION DATA

| | | | | DOSE PER TREATMENT | | |
|---|---|---|---|---|---|---|
| | | TOTAL SCORE | | KETAMINE | OXYTOCIN | TORADOL |
| Treatment | Day | (0-27) | SEVERITY | (mg/kg) | (IU) | (mg) |
| 1 | 0 | 18 | SD | 0.78 | 0 | 0 |
| 2 | 4 | 12 | SD | 1.17 | 24 | 0 |
| 3 | 7 | 10 | SD | 1.56 | 24 | 0 |
| 4 | 11 | 11 | SD | 2.03 | 24 | 0 |
| 5 | 14 | 9 | SD | 2.34 | 24 | 0 |
| 6 | 22 | 3 | SD | 2.34 | 24 | 0 |
| 7 | 27 | 9 | SD | 2.34 | 24 | 30 |
| 8 | 34 | 12 | SD | 2.34 | 24 | 30 |
| 9 | 46 | 8 | SD | 2.34 | 24 | 30 |
| 10 | 70 | 2 | SD | 2.34 | 24 | 30 |
| 11 | 96 | 2 | SD | 2.34 | 24 | 0 |
| 12 | 120 | 4 | SD | 2.34 | 24 | 30 |
| 13 | 141 | 7 | SD | 2.34 | 24 | 30 |

BURNS ANXIETY INVENTORY DATA

| | | | | ANXIOUS | ANXIOUS | PHYSICAL |
|---|---|---|---|---|---|---|
| | | TOTAL | | FEELINGS | THOUGHTS | SYMPTOMS |
| Treatment | Day | SCORE (0-99) | SEVERITY | (0-18) | (0-33) | (0-48) |
| 1 | 0 | 38 | SEVERE | 8 | 15 | 15 |
| 2 | 4 | 30 | MODERATE | 6 | 12 | 12 |
| 3 | 7 | 25 | MODERATE | 5 | 11 | 9 |
| 4 | 11 | 22 | MODERATE | 6 | 10 | 6 |
| 5 | 14 | 16 | MILD | 4 | 8 | 4 |
| 6 | 22 | 7 | BORDERLINE | 2 | 4 | 1 |
| 7 | 27 | 15 | MILD | 5 | 9 | 4 |
| 8 | 34 | 19 | MILD | 5 | 8 | 6 |
| 9 | 46 | 11 | MILD | 3 | 4 | 4 |
| 10 | 70 | 9 | BORDERLINE | 2 | 5 | 2 |
| 11 | 96 | 9 | BORDERLINE | 1 | 6 | 2 |
| 12 | 120 | 4 | MINIMAL | 2 | 1 | 1 |
| 13 | 141 | 10 | BORDERLINE | 3 | 5 | 2 |

Patient 5: The patient was experiencing severe depression and mild psychoses. Current medication regimen was not helping. Meds: Latuda 120 mg; Seroquel 25 mg at night. The patient entered the positive zone after treatment 5. The patient weaned off her Latuda and Seroquel after treatments and has been maintaining a job and romantic relationship.

PHQ-9 DEPRESSION DATA

| | | | | DOSE PER TREATMENT | | |
|---|---|---|---|---|---|---|
| | | TOTAL | | KETAMINE | OXYTOCIN | TORADOL |
| Treatment | Day | SCORE (0-27) | SEVERITY | (mg/kg) | (IU) | (mg) |
| 1 | 0 | 18 | ED | 0.46 | 24 | 0 |
| 2 | 3 | 20 | ED | 0.69 | 24 | 0 |
| 3 | 7 | 15 | VD | 0.84 | 24 | 0 |
| 4 | 10 | 16 | ED | 1.00 | 24 | 0 |
| 5 | 14 | 14 | VD | 1.08 | 24 | 0 |
| 6 | 17 | 11 | SD | 1.15 | 24 | 0 |
| 7 | 23 | 9 | SD | 1.08 | 24 | 0 |
| 8 | 32 | 6 | SD | 1.08 | 24 | 0 |
| 9 | 42 | 6 | SD | 0.00 | 24 | 0 |
| 10 | 49 | 15 | VD | 1.08 | 24 | 0 |
| 11 | 65 | 5 | SD | 1.08 | 24 | 30 |

BURNS ANXIETY INVENTORY DATA

| | | | | ANXIOUS | ANXIOUS | PHYSICAL |
|---|---|---|---|---|---|---|
| | | TOTAL | | FEELINGS | THOUGHTS | SYMPTOMS |
| Treatment | Day | SCORE (0-99) | SEVERITY | (0-18) | (0-33) | (0-48) |
| 1 | 0 | 43 | SEVERE | 13 | 14 | 16 |
| 2 | 3 | 51 | EXTREME | 13 | 18 | 20 |
| 3 | 7 | 43 | SEVERE | 12 | 16 | 15 |
| 4 | 10 | 34 | SEVERE | 11 | 9 | 14 |
| 5 | 14 | 38 | SEVERE | 13 | 9 | 16 |
| 6 | 17 | 30 | MODERATE | 8 | 6 | 16 |
| 7 | 23 | 26 | MODERATE | 6 | 9 | 11 |
| 8 | 32 | 24 | MODERATE | 6 | 7 | 11 |
| 9 | 42 | 22 | MODERATE | 4 | 7 | 12 |
| 10 | 49 | 32 | SEVERE | 10 | 11 | 11 |
| 11 | 65 | 13 | MILD | 4 | 4 | 5 |

Patient 6: Patient was just taking supplements and did not want to take any oral anti-depressant medications. The patient entered the positive zone after treatment 2. Patient hasn't needed any since last treatment.

PHQ-9 DEPRESSION DATA

| | | | | DOSE PER TREATMENT | | |
|---|---|---|---|---|---|---|
| | | TOTAL SCORE | | KETAMINE | OXYTOCIN | TORADOL |
| Treatment | Day | (0-27) | SEVERITY | (mg/kg) | (IU) | (mg) |
| 1 | 0 | 14 | VD | 0.56 | 24 | 0 |
| 2 | 5 | 7 | SD | 0.59 | 24 | 0 |
| 3 | 8 | 6 | SD | 0.68 | 24 | 0 |
| 4 | 13 | 7 | SD | 0.76 | 24 | 0 |
| 5 | 16 | 3 | SD | 0.76 | 24 | 0 |
| 6 | 21 | 1 | ND | 0.76 | 24 | 0 |
| 7 | 49 | 3 | ND | 0.76 | 24 | 30 |

BURNS ANXIETY INVENTORY DATA

| | | | | ANXIOUS | ANXIOUS | PHYSICAL |
|---|---|---|---|---|---|---|
| | | TOTAL | | FEELINGS | THOUGHTS | SYMPTOMS |
| Treatment | Day | SCORE (0-99) | SEVERITY | (0-18) | (0-33) | (0-48) |
| 1 | 0 | 50 | SEVERE | 11 | 22 | 17 |
| 2 | 5 | 34 | SEVERE | 6 | 14 | 14 |
| 3 | 8 | 14 | MILD | 3 | 8 | 3 |
| 4 | 13 | 18 | MILD | 3 | 11 | 4 |
| 5 | 16 | 10 | BORDERLINE | 1 | 4 | 5 |
| 6 | 21 | 8 | BORDERLINE | 3 | 3 | 2 |
| 7 | 49 | 15 | MILD | 4 | 6 | 5 |

Patient 10: This patient had PTSD starting at the age of 17, and struggled off and on with depression and anxiety until experiencing a new traumatic event approx. 1 year prior to treatment. Meds: Lexapro 20 mg once daily; Xanax 0.5 mg as needed. The patients anxiety responded much quicker with oxytocin than what was seen with ketamine treatment alone.

PHQ-9 DEPRESSION DATA

| | | | | DOSE PER TREATMENT | | |
|---|---|---|---|---|---|---|
| | | TOTAL | | KETAMINE | OXYTOCIN | TORADOL |
| Treatment | Day | SCORE (0-27) | SEVERITY | (mg/kg) | (IU) | (mg) |
| 1 | 0 | 10 | VD | 0.43 | 0 | 0 |
| 2 | 3 | 11 | SD | 0.70 | 24 | 0 |
| 3 | 7 | 7 | SD | 0.78 | 24 | 0 |
| 4 | 10 | 9 | SD | 0.96 | 24 | 0 |
| 5 | 15 | 7 | SD | 1.14 | 24 | 0 |
| 6 | 28 | 3 | ND | 1.05 | 24 | 0 |
| 7 | 50 | 4 | SD | 1.14 | 24 | 0 |
| 8 | 79 | 3 | ND | 1.14 | 24 | 0 |
| 9 | 86 | 2 | ND | 0.00 | 0 | 0 |
| 10 | 190 | 3 | ND | 0.00 | 0 | 0 |

BURNS ANXIETY INVENTORY DATA

| | | | | ANXIOUS | ANXIOUS | PHYSICAL |
|---|---|---|---|---|---|---|
| | | TOTAL | | FEELINGS | THOUGHTS | SYMPTOMS |
| Treatment | Day | SCORE (0-99) | SEVERITY | (0-18) | (0-33) | (0-48) |
| 1 | 0 | 49 | SEVERE | 13 | 21 | 15 |
| 2 | 3 | 28 | MODERATE | 7 | 14 | 7 |
| 3 | 7 | 14 | MILD | 3 | 8 | 3 |
| 4 | 10 | 18 | MILD | 4 | 9 | 5 |
| 5 | 15 | 12 | MILD | 4 | 4 | 4 |
| 6 | 28 | 6 | BORDERLINE | 3 | 2 | 1 |
| 7 | 50 | 6 | BORDERLINE | 3 | 2 | 1 |
| 8 | 79 | 9 | BORDERLINE | 3 | 3 | 3 |
| 9 | 86 | 3 | MINIMAL | 2 | 1 | 0 |
| 10 | 190 | 12 | MILD | 3 | 4 | 5 |

Patient 11: Patient had tried two anti-depressants without any relief. The patient's current medication regimen was also not helping. Current Meds: Adderall (extended release) 30 mg once daily; Adderall (immediate release) 10 mg up to 3 times a day as needed for concentration and focus; Lexapro 20 mg once daily; trazadone 50 mg at night; Nexium as needed and Allegra as needed. Patient responded well and was in the positive zone after treatment 4. The patient had some significant stressors as she first got into the positive zone. The patient hasn't needed any treatments since her last treatment.

PHQ-9 DEPRESSION DATA

| | | | | DOSE PER TREATMENT | | |
|---|---|---|---|---|---|---|
| | | TOTAL | | KETAMINE | OXYTOCIN | TORADOL |
| Treatment | Day | SCORE (0-27) | SEVERITY | (mg/kg) | (IU) | (mg) |
| 1 | 0 | 23 | ED | 0.62 | 0 | 0 |
| 2 | 4 | 17 | SD | 0.74 | 24 | 0 |
| 3 | 7 | 8 | SD | 0.79 | 24 | 0 |
| 4 | 11 | 9 | SD | 0.85 | 24 | 0 |
| 5 | 14 | 14 | SD | 0.91 | 24 | 0 |
| 6 | 18 | 11 | SD | 1.02 | 24 | 0 |
| 7 | 34 | 8 | SD | 1.02 | 24 | 0 |
| 8 | 67 | 7 | SD | 1.02 | 24 | 30 |

BURNS ANXIETY INVENTORY DATA

| | | | | ANXIOUS | ANXIOUS | PHYSICAL |
|---|---|---|---|---|---|---|
| | | TOTAL | | FEELINGS | THOUGHTS | SYMPTOMS |
| Treatment | Day | SCORE (0-99) | SEVERITY | (0-18) | (0-33) | (0-48) |
| 1 | 0 | 41 | SEVERE | 12 | 13 | 16 |
| 2 | 4 | 26 | MODERATE | 11 | 5 | 10 |
| 3 | 7 | 20 | MILD | 5 | 3 | 12 |
| 4 | 11 | 14 | MILD | 3 | 2 | 9 |
| 5 | 14 | 18 | MILD | 6 | 2 | 10 |
| 6 | 18 | 26 | MODERATE | 6 | 3 | 17 |
| 7 | 34 | 19 | MILD | 6 | 2 | 11 |
| 8 | 67 | 10 | BORDERLINE | 3 | 1 | 6 |

Patient 14: Patient did not want to take any oral anti-depressants as they haven't helped in the past. Patient got into the positive zone after treatment 3. The patient did great after his last treatment and hasn't needed anymore.

PHQ-9 DEPRESSION DATA

| | | | | DOSE PER TREATMENT | | |
|---|---|---|---|---|---|---|
| | | TOTAL SCORE | | KETAMINE | OXYTOCIN | TORADOL |
| Treatment | Day | (0-27) | SEVERITY | (mg/kg) | (IU) | (mg) |
| 1 | 0 | 21 | VD | 0.74 | 24 | 30 |
| 2 | 3 | 14 | SD | 0.99 | 24 | 30 |
| 3 | 6 | 16 | SD | 1.05 | 24 | 30 |
| 4 | 10 | 4 | ND | 1.05 | 24 | 30 |
| 5 | 13 | 3 | ND | 1.12 | 24 | 30 |
| 6 | 19 | 3 | ND | 1.12 | 0 | 30 |

BURNS ANXIETY INVENTORY DATA

| | | | | ANXIOUS | ANXIOUS | PHYSICAL |
|---|---|---|---|---|---|---|
| | | TOTAL | | FEELINGS | THOUGHTS | SYMPTOMS |
| Treatment | Day | SCORE (0-99) | SEVERITY | (0-18) | (0-33) | (0-48) |
| 1 | 0 | 39 | SEVERE | 9 | 12 | 18 |
| 2 | 3 | 16 | MILD | 2 | 6 | 8 |
| 3 | 6 | 23 | MODERATE | 3 | 5 | 15 |
| 4 | 10 | 10 | BORDERLINE | 0 | 3 | 7 |
| 5 | 13 | 8 | BORDERLINE | 0 | 1 | 7 |
| 6 | 19 | 7 | BORDERLINE | 0 | 2 | 5 |

Patient 15: Patient had chronic pain, depression, and anxiety. Patient on high dose opioids and wanted to wean down on the doses, but the depression and anxiety were too much when weaning. The patient felt his oral anti-depressant medications hadn't helped in the past. Meds: Fentanyl patch 75 mcg every 2 days; Oxycodone 30 mg tablets taken 3-5 times per day as needed. The patient was in the positive zone after treatment 4.

PHQ-9 DEPRESSION DATA

| | | | | DOSE PER TREATMENT | | |
|---|---|---|---|---|---|---|
| | | TOTAL | | KETAMINE | OXYTOCIN | TORADOL |
| Treatment | Day | SCORE (0-27) | SEVERITY | (mg/kg) | (IU) | (mg) |
| 1 | 0 | 14 | VD | 0.49 | 24 | 30 |
| 2 | 2 | 0 | ND | 0.66 | 24 | 30 |
| 3 | 6 | 0 | ND | 0.81 | 24 | 30 |
| 4 | 8 | 4 | SD | 0.96 | 24 | 30 |
| 5 | 13 | 7 | SD | 1.11 | 24 | 30 |
| 6 | 16 | 5 | ND | 1.26 | 24 | 30 |
| 7 | 21 | 8 | SD | 1.41 | 24 | 30 |
| 8 | 28 | 4 | SD | 1.31 | 24 | 30 |
| 9 | 42 | 10 | SD | 1.31 | 24 | 30 |
| 10 | 56 | 4 | SD | 1.31 | 24 | 30 |
| 11 | 70 | 2 | ND | 1.31 | 24 | 30 |
| 12 | 93 | 3 | ND | 1.31 | 24 | 30 |

BURNS ANXIETY INVENTORY DATA

| | | | | ANXIOUS | ANXIOUS | PHYSICAL |
|---|---|---|---|---|---|---|
| | | TOTAL | | FEELINGS | THOUGHTS | SYMPTOMS |
| Treatment | Day | SCORE (0-99) | SEVERITY | (0-18) | (0-33) | (0-48) |
| 1 | 0 | 32 | SEVERE | 8 | 5 | 19 |
| 2 | 2 | 0 | MIN | 0 | 0 | 0 |
| 3 | 6 | 2 | MIN | 2 | 0 | 0 |
| 4 | 8 | 16 | MILD | 6 | 6 | 4 |
| 5 | 13 | 12 | MILD | 4 | 2 | 6 |
| 6 | 16 | 7 | BORDERLINE | 5 | 1 | 1 |
| 7 | 21 | 14 | MILD | 4 | 6 | 4 |
| 8 | 28 | 12 | MILD | 6 | 4 | 2 |
| 9 | 42 | 13 | MILD | 6 | 6 | 1 |
| 10 | 56 | 6 | BORDERLINE | 5 | 0 | 1 |
| 11 | 70 | 3 | MIN | 3 | 0 | 0 |
| 12 | 93 | 16 | MILD | 6 | 4 | 6 |

Patient 16: Patient had chronic pain from a devastating car accident which caused a traumatic brain injury and an L5 burst fracture. The patient had struggled with depression since 12 years old. The patient didn't have any success with oral anti-depressants and didn't want to take them. Meds: Spironolactone for acne. The patient was in the positive zone after treatment 2. The patient had difficulty adjusting to the positive zone but is doing much better with pain, depression and anxiety.

PHQ-9 DEPRESSION DATA

| | | | | DOSE PER TREATMENT | | |
|---|---|---|---|---|---|---|
| | | TOTAL | | KETAMINE | OXYTOCIN | TORADOL |
| Treatment | Day | SCORE (0-27) | SEVERITY | (mg/kg) | (IU) | (mg) |
| 1 | 0 | 21 | ED | 0.55 | 0 | 30 |
| 2 | 4 | 16 | VD | 0.73 | 24 | 30 |
| 3 | 8 | 16 | VD | 0.83 | 24 | 30 |
| 4 | 12 | 11 | SD | 0.83 | 24 | 30 |
| 5 | 15 | 13 | SD | 0.83 | 24 | 30 |
| 6 | 19 | 9 | SD | 0.83 | 24 | 30 |
| 7 | 26 | 12 | SD | 0.83 | 24 | 30 |
| 8 | 55 | 7 | SD | 0.83 | 24 | 30 |
| 9 | 84 | 9 | SD | 0.83 | 24 | 30 |

BURNS ANXIETY INVENTORY DATA

| | | | | ANXIOUS | ANXIOUS | PHYSICAL |
|---|---|---|---|---|---|---|
| | | TOTAL | | FEELINGS | THOUGHTS | SYMPTOMS |
| Treatment | Day | SCORE (0-99) | SEVERITY | (0-18) | (0-33) | (0-48) |
| 1 | 0 | 57 | EXTREME | 13 | 21 | 23 |
| 2 | 4 | 45 | SEVERE | 15 | 20 | 10 |
| 3 | 8 | 43 | SEVERE | 9 | 24 | 10 |
| 4 | 12 | 43 | SEVERE | 11 | 18 | 14 |
| 5 | 15 | 38 | SEVERE | 11 | 16 | 11 |
| 6 | 19 | 32 | SEVERE | 4 | 16 | 12 |
| 7 | 26 | 36 | SEVERE | 7 | 20 | 9 |
| 8 | 55 | 29 | MODERATE | 6 | 13 | 10 |
| 9 | 84 | 35 | SEVERE | 8 | 18 | 9 |

Patient 18: The patient got out of the mental health ward the same day as the first treatment. The patient had been doing self-harm by cutting. This patient was deep in the negative zone and didn't want to be on anti-depressants. Meds: none. The patient entered into the positive zone after treatment 4. The patient started Buspar 10 mg twice daily and Lexapro 10 mg once daily after treatment 2 as wasn't sure if they could continue with the ketamine treatments. The patient was very happy with his results and he felt more resilient despite his significant stressors.

PHQ-9 DEPRESSION DATA

| | | | | DOSE PER TREATMENT | | |
|---|---|---|---|---|---|---|
| | | TOTAL SCORE | | KETAMINE | OXYTOCIN | TORADOL |
| Treatment | Day | (0-27) | SEVERITY | (mg/kg) | (IU) | (mg) |
| 1 | 0 | 27 | EXT | 0.55 | 25 | 30 |
| 2 | 3 | 12 | EXT | 0.73 | 25 | 30 |
| 3 | 6 | 20 | EXT | 0.73 | 25 | 30 |
| 4 | 10 | 27 | EXT | 1.00 | 25 | 30 |
| 5 | 13 | 16 | EXT | 1.00 | 25 | 30 |
| 6 | 21 | 21 | VD | 1.00 | 25 | 30 |

BURNS ANXIETY INVENTORY DATA PHYSICAL

| | | | | ANXIOUS | ANXIOUS | PHYSICAL |
|---|---|---|---|---|---|---|
| | | TOTAL SCORE | | FEELINGS | THOUGHTS | SYMPTOMS |
| Treatment | Day | (0-99) | SEVERITY | (0-18) | (0-33) | (0-48) |
| 1 | 0 | 78 | EXTREME | 18 | 24 | 36 |
| 2 | 3 | 44 | SEVERE | 9 | 17 | 18 |
| 3 | 6 | 34 | SEVERE | 9 | 8 | 17 |
| 4 | 10 | 62 | EXTREME | 15 | 24 | 23 |
| 5 | 13 | 43 | SEVERE | 9 | 17 | 17 |
| 6 | 21 | 33 | SEVERE | 8 | 13 | 12 |

Patient 19: This patient had been struggling with depression and anxiety since teenage years. The patient had tried anti-depressants over the years with not much relief. Current Meds: Celexa 60 mg once daily; Armor thyroid 90 mg once daily. The patient entered the positive zone after treatment 3. The patient has continued to do well, and planning to wean off Celexa.

PHQ-9 DEPRESSION DATA

| | | | | DOSE PER TREATMENT | | |
|---|---|---|---|---|---|---|
| | | TOTAL SCORE | | KETAMINE | OXYTOCIN | TORADOL |
| Treatment | Day | (0-27) | SEVERITY | (mg/kg) | (IU) | (mg) |
| 1 | 0 | 18 | VD | 0.42 | 0 | 30 |
| 2 | 2 | 7 | ND | 0.53 | 24 | 0 |
| 3 | 5 | 6 | ND | 0.53 | 24 | 30 |
| 4 | 8 | 3 | ND | 0.59 | 24 | 30 |
| 5 | 12 | 2 | ND | 0.59 | 24 | 30 |
| 6 | 15 | 2 | ND | 0.59 | 24 | 30 |
| 7 | 21 | 6 | SD | 0.59 | 24 | 30 |
| 8 | 27 | 3 | ND | 0.59 | 24 | 30 |
| 9 | 55 | 4 | ND | 0.59 | 24 | 30 |

BURNS ANXIETY INVENTORY DATA

| | | | | ANXIOUS | ANXIOUS | PHYSICAL |
|---|---|---|---|---|---|---|
| | | TOTAL | | FEELINGS | THOUGHTS | SYMPTOMS |
| Treatment | Day | SCORE (0-99) | SEVERITY | (0-18) | (0-33) | (0-48) |
| 1 | 0 | 32 | MILD | 5 | 9 | 18 |
| 2 | 2 | 15 | MILD | 4 | 1 | 10 |
| 3 | 5 | 14 | MILD | 2 | 0 | 12 |
| 4 | 8 | 11 | MILD | 0 | 0 | 11 |
| 5 | 12 | 6 | BORD | 0 | 0 | 6 |
| 6 | 15 | 8 | BORD | 0 | 0 | 8 |
| 7 | 21 | 4 | MIN | 0 | 0 | 4 |
| 8 | 27 | 2 | MIN | 0 | 0 | 2 |
| 9 | 55 | 2 | MIN | 1 | 0 | 1 |

Tables 3 and 4 show patient information and percent improvement of symptoms achieved using the methods described herein after treatments 3, 4, 5, and 6. Although it may be that a patient is able to move from the acute “stabilization” phase to the maintenance phase in 4-5 treatments (or 2-3 weeks), it may vary based on the particular patient.

TABLE 3
PHQ-9

| | | % | % | % | % |
|---|---|---|---|---|---|
| | | improve- | improve- | improve- | improve- |
| | | ment | ment | ment | ment |
| | | after | after | after | after |
| Patient | | treatment | treatment | treatment | treatment |
| No. | Diagnosis | 3* | 4* | 5* | 6* |
| 1 | Depression, | 85% | 93% | 89% | 93% |
| | Anxiety, and | | | | |
| | PTSD | | | | |
| 2 | Anxiety, | 53% | 35% | 35% | 53% |
| | Depression, | | | | |
| | and OCD | | | | |
| 3 | Bipolar 1, | 68% | 95% | 100% | 95% |
| | Alcohol use | | | | |
| | Disorder, and | | | | |
| | Depression | | | | |
| 4 | OCD and | 39% | 50% | 83% | 50% |
| | Depression | | | | |
| 5 | Bipolar 1 and | 11% | 22% | 39% | 50% |
| | Schizoaffective | | | | |
| | Disorder | | | | |
| 6 | Depression | 50% | 79% | 93% | 79% |
| | and Anxiety | | | | |
| 10 | PTSD, | 10% | 30% | 70% | 60% |
| | Depression | | | | |
| | and Anxiety | | | | |
| 11 | Depression, | 61% | 39% | 52% | 65% |
| | ADHD, and | | | | |
| | Anxiety | | | | |
| 14 | Depression | 81% | 86% | 86% | ND |
| | and Gambling | | | | |
| | Addiction | | | | |
| 15 | Depression | 71% | 50% | 64% | 43% |
| | and Anxiety | | | | |
| 16 | Depression, | 48% | 38% | 57% | 43% |
| | Anxiety and | | | | |
| | Chronic Pain | | | | |
| 18 | Depression, | 63% | 52% | 22% | ND |
| | Anxiety, | | | | |
| | Borderline | | | | |
| | Personality | | | | |
| | Disorder | | | | |
| 19 | Depression | 83% | 89% | 89% | 67% |
| | and Anxiety | | | | |

*based on Total Score
ND = No Data

TABLE 4
Burn Anxiety Inventory

| | | % | % | % | % |
|---|---|---|---|---|---|
| | | improve- | improve- | improve- | improve- |
| | | ment | ment | ment | ment |
| | | after | after | after | after |
| Patient | | treatment | treatment | treatment | treatment |
| No. | Diagnosis | 3* | 4* | 5* | 6* |
| 1 | Depression, | 87% | 87% | 92% | 87% |
| | Anxiety, and | | | | |
| | PTSD | | | | |
| 2 | Anxiety, | 34% | 30% | 30% | 34% |
| | Depression, and | | | | |
| | OCD | | | | |
| 3 | Bipolar 1, | 61% | 94% | 100% | 97% |
| | Alcohol use | | | | |
| | Disorder, and | | | | |
| | Depression | | | | |
| 4 | OCD and | 42% | 58% | 92% | 61% |
| | Depression | | | | |
| 5 | Bipolar 1 and | 21% | 12% | 30% | 40% |
| | Schizoaffective | | | | |
| | Disorder | | | | |
| 6 | Depression and | 64% | 80% | 84% | 70% |
| | Anxiety | | | | |
| 10 | PTSD, | 63% | 76% | 88% | 88% |
| | Depression and | | | | |
| | Anxiety | | | | |
| 11 | Depression, | 66% | 56% | 37% | 54% |
| | ADHD, and | | | | |
| | Anxiety | | | | |
| 14 | Depression and | 74% | 79% | 82% | ND |
| | Gambling | | | | |
| | Addiction | | | | |
| 15 | Depression and | 50% | 62% | 78% | 56% |
| | Anxiety | | | | |
| 16 | Depression, | 25% | 33% | 44% | 37% |
| | Anxiety and | | | | |
| | Chronic Pain | | | | |
| 18 | Depression, | 80% | 45% | 58% | ND |
| | Anxiety, | | | | |
| | Borderline | | | | |
| | Personality | | | | |
| | Disorder | | | | |
| 19 | Depression and | 66% | 81% | 75% | 88% |
| | Anxiety | | | | |

*based on Total Score
ND = No Data
* * *

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A method for treating one or more disease or disorders selected from severe anxiety, major depressive disorder (MDD) single or recurrent, persistent depressive disorder (cyclothymia), treatment resistant depression (TRD), disruptive mood regulation disorder, bipolar 1 disorder, bipolar 2 disorder, premenstrual dysphoric disorder, schizoaffective disorder, adjustment disorder, complex regional pain syndrome (CRPS) type 1, CRPS type 2, chronic neuropathic pain, chronic pain syndromes, chronic low back pain, fibromyalgia, migraine headaches, chronic neuropathic pain, acute neuropathic pain, or irritable bowel syndrome, in a subject, comprising administering an effective amount of oxytocin, an effective amount of a neuroplasticity agent, and optionally an effective amount of an anti-inflammatory agent, to a subject in need thereof.

2. The method of claim 1, wherein the neuroplasticity agent is one or more of racemic ketamine, esketamine, (R)-ketamine, (2R,6R)-hydroxynorketamine (HNK), psilocybin, 3,4-methylenedioxy methamphetamine (MDMA), N,N-dimethyltryptamine (DMT or N,N-DMT), lysergic acid diethylamide (LSD), dextromethorphan, nuedexta (a combination of dextromethorphan and quinidine), deudextromethorphan (AVP-786), axsome (AXS-05), dextromethadone (REL-1017), or zuranolone (SAGE-217).

3. A method for treating post-partum depression or peripartum depression, in a subject, comprising administering an effective amount of oxytocin, an effective amount of a neuroplasticity agent and an effective amount of an anti-inflammatory agent, to a subject in need thereof.

4. The method of claim 3, wherein the neuroplasticity agent is brexanolone or zuranolone (SAGE-217).

5. The method of any preceding claim, wherein the method comprises an acute phase and a maintenance phase.

6. The method of any preceding claim, wherein the acute phase comprises administering a first dose of oxytocin prior to administering the neuroplasticity agent.

7. The method of any preceding claim, wherein the acute phase comprises administering a first dose of the neuroplasticity agent prior to administering oxytocin.

8. The method of any one of claims 1-7, wherein the oxytocin is administered intravenously.

9. The method of any one of claims 1-7, wherein the oxytocin is administered intranasally.

10. The method of any one of claims 1-9, wherein the neuroplasticity agent is administered intravenously.

11. The method of any one of claims 1-9, wherein the neuroplasticity agent is administered intranasally.

12. The method of any one of claims 1-11, wherein the dose of oxytocin is 0.1 to 100 International Units (IU).

13. The method of any one of claims 1-12, wherein the acute phase comprises administering racemic ketamine, esketamine, or (R)-ketamine.

14. The method of any one of claims 1-13, wherein the acute phase comprises administering racemic ketamine, esketamine, or (R)-ketamine intravenously at a total dose of about 60 mg, optionally over 50 minutes.

15. The method of any one of claims 1-13, wherein the acute phase comprises administering racemic ketamine, esketamine, or (R)-ketamine intranasally at an initial dose of 0.1 to 300 mg, optionally with a second intranasal ketamine dose administered 10-15 minutes later at a dose of 0.1 to 300 mg.

16. The method of any one of claims 1-15, wherein the acute phase is 2 treatments per week for 3-5 weeks.

17. The method of any one of claims 1-16, wherein the maintenance phase comprises one treatment per week for 2-4 weeks, then one treatment every other week, or one treatment every 2-4 weeks for the first 6 months.

18. The method of any one of claims 1-17, wherein each treatment dose of the maintenance phase decreases, remain unchanged, or increases from the previous treatment.

19. The method of any one of claims 1-18, wherein each treatment dose of the maintenance phase increases 5 mg, 10 mg, or 15 mg based on the patient's response from the previous treatment.

20. A method for treating generalized anxiety disorder (GAD), panic disorder, post-traumatic stress disorder, separation anxiety disorder, obsessive compulsive disorder (OCD), social anxiety disorder, body dysmorphic disorder, anorexia nervosa, bulimia nervosa, binge eating disorder, in a subject, comprising administering an effective amount of oxytocin and an effective amount of a neuroplasticity agent to a subject in need thereof.

21. The method of claim 20, wherein the subject is from 16-18 years or from 66-75 years.

22. The method of claim 20 or 21, wherein the method comprises an acute phase and a maintenance phase.

23. The method of any one of claims 20-22, wherein the acute phase comprises administering a first dose of oxytocin prior to administering the neuroplasticity agent.

24. The method of any one of claims 20-23, wherein the acute phase comprises administering a first dose of the neuroplasticity agent prior to administering oxytocin.

25. The method of any one of claims 20-24, wherein the oxytocin is administered intravenously.

26. The method of any one of claims 20-25, wherein the oxytocin is administered intranasally.

27. The method of any one of claims 20-26, wherein the neuroplasticity agent is administered intravenously.

28. The method of any one of claims 20-26, wherein the neuroplasticity agent is administered intranasally.

29. The method of any one of claims 20-28, wherein the dose of oxytocin is 0.1 to 75 International Units (IU).

30. The method of any one of claims 20-29, wherein the acute phase comprises administering racemic ketamine, esketamine, or (R)-ketamine.

31. The method of any one of claims 20-30, wherein the acute phase comprises administering racemic ketamine, esketamine, or (R)-ketamine intravenously at a total dose of about 40 mg, optionally over 50 minutes.

32. The method of any one of claims 20-31, wherein the acute phase comprises administering racemic ketamine, esketamine, or (R)-ketamine intranasally at an initial dose of 0.1 to 200 mg, optionally with a second intranasal ketamine dose administered 10-15 minutes later at a dose of 0.1 to 200 mg.

33. The method of any one of claims 20-32, wherein the acute phase is 2 treatments per week for 3-5 weeks.

34. The method of any one of claims 20-33, wherein the maintenance phase comprises one treatment per week for 2-4 weeks, then one treatment every other week, or one treatment every 2-4 weeks for the first 6 months.

35. The method of any one of claims 20-34, wherein each treatment dose of the maintenance phase decreases, remain unchanged, or increases from the previous treatment.

36. The method of any one of claims 20-35, wherein each treatment dose of the maintenance phase increases 5 mg, 10 mg, or 15 mg based on the patient's response from the previous treatment.

37. The method of any one of claims 1-36, wherein the method further comprises administering an anti-inflammatory agent.

38. The method of any one of claims 1-36, wherein the method further comprises administering a nonsteroidal anti-inflammatory drug (NSAID).

39. The method of any one of claims 1-36, wherein the method further comprises administering ketorolac.
